(19) United States
(12) Patent Application Publication
(10) Pub. No.: US 2019/0134169 A1
(43) Pub. Date: May 9, 2019

(54) DLL4-expressing cells and vaccine using the same

(71) Applicant: Temple University-Of The Commonwealth System of Higher Education, Philadelphia, PA (US)

(72) Inventors: Yi Zhang, Wallingford, PA (US); Lijun Meng, Philadelphia, PA (US); Shan He, Blue Bell, PA (US)

(73) Assignee: Temple University-Of The Commonwealth System of Higher Education, Philadeiphia, PA (US)

(21) Appl. No.: 16/095,986

(22) PCT Filed: Apr. 26, 2017
(86) PCT No.: PCT/US17/29601

§ 371 Date: Oct. 24, 2018

Related U.S. Application Data
(60) Provisional application No. 62/327,599, filed on Apr. 26, 2016.

(51) Int. Cl. A61K 39/00; A61P 37/06; A61P 35/00; A61K 39/02; A61K 39/12

(52) U.S. Cl. CPC A61K 39/001; A61P 37/06; A61K 39/0002; A61K 39/02; A61K 39/12; A61P 35/00

(57) ABSTRACT

Methods are provided for generating DLL4-expressing immune cells. The invention also includes cellular compositions of dendritic and T cells produced by these methods. The immune cells of the invention can be used widely as components in many diagnostic and therapeutic systems, including improved vaccines to reduce the risk of graft versus host disease.

### CROSS REFERENCE TO RELATED APPLICATIONS

This application claims priority to U.S. Provisional Application No. 62/327,599, filed Apr. 26, 2016 which is hereby incorporated by reference herein in its entirety.

### STATEMENT REGARDING FEDERALLY SPONSORED RESEARCH OR DEVELOPMENT

This invention was made with government support under 1R01CA172106-01 awarded by the National Institutes of Health. The government has certain rights in the invention.

### BACKGROUND OF THE INVENTION

Cancer research has seen significant advances that have led to the steady reduction in mortality rates for many types of malignancies. This reduction in mortality rate has been influenced by improvements in early detection, advanced surgical techniques and the employment of novel therapeutic interventions. Given the success in decreasing cancer-related mortality rates, there has been a shift in research to focus on novel targeted therapies against cancer, of which the development of vaccines has been in the forefront. Vaccines have been highly effective in reducing mortality from pathogens due to their ability to activate the immune system and render immunity to foreign antigens. Not only does effective vaccination facilitate reduction in mortality, but vaccines also induce long-term immunity that protects against recurrent infection.

Currently, methods used for immunotherapy typically incorporate ex vivo stimulated antigen presenting cells (APC), where dendritic cell (DC) precursors are cultured immediately after collection from the patient, and then antigen loaded DCs are infused into the patient as soon as they are harvested. DCs have the unique capacity to elicit primary T cell immune responses. DCs process and present Ag peptides, activate naive T cells, and promote activated T cell expansion and survival through the expression of costimulatory molecules. DCs also produce effector-polarizing cytokines that are crucial in directing effective T cell differentiation.

Notch signaling is critical for alloreactive T cell responses. Notch ligands expressed on the surface of DCs are important in promoting the generation of different lineages of effector T cells. Notch ligands (DLL1, DLL 3, DLL4, Jagged1 and Jagged2) interact with Notch receptors (Notch 1, 2, 3, and 4), triggering the release of intracellular Notch and the subsequent transcription of Notch target genes. Inhibiting pan-Notch signaling in donor T cells reduced their production of IFN-γ and IL-17 (Zhang et al., Blood, 2011, 117:299-308). Notch ligand DLL4 mediates a dominant role for activating Notch signaling in alloreactive T cells (Tran et al., J Clin Invest, 2013, 123:1590-604).

A specific type of DCs that express high levels of DLL4 (named DLL4hi DCs have greater ability than DLL4-negative conventional DCs to induce CD4⁺ Th1 and CD8⁺ CTLs. However, monocyte-derived DCs do not naturally express DLL4. Thus, there is a need in the art for efficient and directed means of obtaining a high density population of DLL4-expressing immune cells. The present invention satisfies this need.

### SUMMARY OF THE INVENTION

In one embodiment, the invention relates to a composition comprising a population of genetically modified DLL4-expressing immune cells. In one embodiment, the immune cells comprise a nucleic acid molecule encoding DLL4. In one embodiment, the nucleic acid is in vitro transcribed RNA.

In one embodiment, the immune cells are selected from the group consisting of immature dendritic cells, mature dendritic cells, activated dendritic cells, T cells, natural killer T (NKT) cells and chimeric antigen receptor (CAR) T cells. In one embodiment, the immune cells are CAR T cells comprising a nucleic acid molecule encoding DLL4 and a nucleic acid molecule encoding a CAR.

In one embodiment, the composition is for use in immunotherapy.

In one embodiment, the invention relates to a composition comprising a population of activated T cells for use in immunotherapy, wherein the T cells have been activated by antigen loaded, activated, DLL4-expressing dendritic cells (DCs).

In one embodiment, the invention relates to a method of generating a DLL4-expressing, antigen loaded, activated dendritic cell (DC), comprising the steps of loading at least one antigen into a DC; and activating the DC with Flt3L and at least one TLR. In one embodiment, the DC is a bone marrow derived DC.

In one embodiment, the at least one TLR comprises at least one of LPS, R848 or a combination thereof.

In one embodiment, the antigen is at least one of a tumor antigen and a microbial antigen.

In one embodiment, the invention relates to a method of generating a DLL4-expressing, antigen loaded, activated DC, comprising the steps of loading at least one antigen into a DC; genetically modifying the DC to express DLL4; and activating the DC. In one embodiment, the method of genetically modifying comprises providing a DNA-plasmid-based system or an mRNA-based system encoding DLL4 to the DC. In one embodiment, the system is an expression vector wherein the expression vector is a retroviral vector or a lentiviral vector.

In one embodiment, the antigen is at least one of a tumor antigen and a microbial antigen.

In one embodiment, the DC is a monocyte derived DC.

In one embodiment, the invention relates to a method of eliciting an immune response in a subject, the method comprising administering to the subject an effective amount of a population of DLL4-expressing immune cells. In one embodiment, the immune response is selected from the group consisting of a Th17 immune response and a Th1 immune response.

In one embodiment, the invention relates to a method of providing anti-tumor immunity in a subject, the method comprising: administering to the subject an effective amount of an antigen loaded, activated, DLL4-expressing DC. In one embodiment, the DC is a bone marrow derived DC which has been is activated with Flt3L and at least one TLR agonist. In one embodiment, the at least one TLR comprises at least one of LPS, R848, or a combination thereof. In one embodiment, the DC is genetically modified to express DLL4.

In one embodiment, the invention relates to a method of generating an activated T cell for use in immunotherapy, comprising contacting a naïve T cell with an antigen loaded, activated, DLL4-expressing DC. In one embodiment, the antigen loaded, activated, DLL4-expressing DC is a bone marrow derived DC and which has been activated with Flt3L and at least one TLR agonist. In one embodiment, the at least one TLR comprises at least one of LPS, R848, or a combination thereof. In one embodiment, the DC is genetically modified to express DLL4.

In one embodiment, the invention relates to a method of stimulating a T cell-mediated immune response to a cell population or tissue in a subject, the method comprising: administering to the subject an effective amount of a T cell selected from the group consisting of a genetically modified T cell comprising a nucleic acid sequence encoding DLL4, a DLL4-expressing CAR T cell and an activated T cell wherein the T cell is activated by loading at least one antigen into a DC; genetically modifying the DC to express DLL4; and activating the DC.

In one embodiment, the invention relates to a method of treating cancer in a subject, the method comprising administering to the subject an effective amount of a genetically modified T cell comprising a nucleic acid sequence encoding DLL4, and a nucleic acid sequence encoding a CAR. In one embodiment, the CAR nucleic acid sequence comprises an antigen binding domain nucleic acid sequence. In one embodiment, the antigen binding domain nucleic acid targets CD19.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1, comprising FIGS. 1A through 1E, depicts in vitro generation of DCs expressing high levels of DLL4. Flt3L-DCs were generated by incubating BALB/c mouse BM mononuclear cells in cultures with Flt3L. GM-DCs were induced by culturing c-kit⁺ hematopoietic progenitor cells in the presence of GM-CSF, IL-4 and SCF. After 8 days in culture, cells were collected and cultured in medium containing indicated stimuli for additional 24 hours. FIG. 1A depicts graphs showing the number of CD11c⁺ cells (mean±SD of triplicates). FIG. 1B depicts histograms and a graph shows the percentage of DLL4 on the surface of Flt3L-DCs (mean±SD of triplicates). FIG. 1C depicts real-time RT-PCR analysis of the relative expression of indicated genes in DCs generated in cultures (mean±SD of triplicates). FIG. 1D depicts histograms showing the expression of tested markers on the surface of DCs with or without stimulation of LPS+R848. Representative results of three independent experiments are shown. **: p<0.01. ***: p<0.001. FIG. 1E depicts real-time RT-PCR analysis of the relative expression of indicated genes in DCs generated in cultures (mean±SD of triplicates).

FIG. 2, comprising FIG. 2A through FIGS. 2H, depicts experimental results demonstrating DLL4^{hi}DCs induce effector CD4⁺ T cell differentiation. DLL4^{hi}DCs or GM-DCs were generated from BM of BALB/c mice. For FIGS. 2A through 2D, CD4⁺ TN were purified from B6 mice, labeled with CFSE, and incubated with BALB/c DLL4^{hi}DC at escalating DC:T cell ratios. Five days later, T cells were collected and evaluated. FIG. 2A depicts a measure of T cell proliferation. FIG. 2B depicts a measure of T cell recovery. FIG. 2C depicts dot plots and graphs showing the percentages of IFN-γ- and IL-17-producing cells among proliferating CD4⁺ T cells that expressed low levels of CFSE (CFSE^{low}) (mean±SD of triplicates). FIG. 2D depicts a graph showing in vitro cytotoxic activity of B6 CD4⁺ TN or BALB/c DLL4^{hi}DCs activated B6 CD4⁺ T cells (DC:T cell ratio of 1:4) against A20 leukemia cells. For FIGS. 2E through 2G, CF SE labeled B6 CD4⁺ TN were incubated with BALB/c DLL4^{hi}DCs or GM-DCs with DC:T cell ratio of 1:4. Five days later, T cells were collected to measure and evaluated. FIG. 2E depicts a measure of T cell proliferation. FIG. 2F depicts a measure of T cell recovery. FIG. 2G depicts dot plots and graphs showing the percentages of IFN-γ, IL-17, and TNFα-producing cells among proliferating CD4⁺ T cells that expressed low levels of CFSE (CFSE^{low}) (mean±SD of triplicates). For FIG. 2H, B6 DLL4^{hi}DCs were cultured with CD4⁺ T cells specific to OT-II peptide (OVA₂₃₂₋₂₃₉), which were isolated from T cell receptor (TCR) transgenic OT-II mice in the presence of OT-II peptides (upper panel). BALB/c DLL4^{hi}DCs were cultured with CD4⁺ T cells isolated from WT B6 mice (lower panel). FIG. 2H depicts dot plots and graphs showing the percentages of IFN-γ and IL-17 in the different neutralizing Ab condition (mean±SD of triplicates). Representative data from two independent experiments are shown. *: P<0.05, **: p<0.01. ***: p<0.001.

FIG. 3, comprising FIGS. 3A through 3C, depicts experimental results demonstrating that DLL4^{hi}DC-induced alloreactive T cells have reduced ability to cause GVHD. DLL4^{hi}DCs and GM-DCs were generated from BM of BALB/c mice. After 8 days in culture, DCs were stimulated by LPS and R848 for 24 hours. Then, both DLL4^{hi}DCs and GM-DCs were cultured with allogeneic B6 CD4⁺ T cells for five days (DC:T cell=1:4). Lethally irradiated (8Gy) BALB/c mice were injected with B6 TCD-BM (5.0×10⁶) mixed with or without either naïve or in vitro activated allogeneic CD4⁺ T cells (1.0×10⁶). FIG. 3A depicts analysis of survival, and GVHD clinical score of the recipients, monitored over time (mean±SEM). Data shown here are pooled from two independent experiments. FIG. 3B depicts representative images showing the skin, liver, and small intestine from one of 6 recipients in each group at day 14 after transplantation. Photographs were obtained with an Olympus BX41 microscope (10/0.3 NA lens, 200× magnification, digital DP70 camera). FIG. 3C depicts graphs showing the pathological scores of GVHD 14 days after HSCT (6 mice per group). **: P<0.01, ***: p<0.001.

FIG. 4, comprising FIGS. 4A through 4F, depicts experimental results showing DLL4^{hi}DC-induced T cells retain potent anti-leukemia activity. DLL4^{hi}DCs were generated from BM of BALB/c mice. Then, the DLL4^{hi}DCs were cultured with allogeneic B6 CD4⁺ T cells for five days (DC:T cell=1:4). Lethally irradiated (8Gy) BALB/c mice were injected with B6 TCD-BM (5.0×10⁶) mixed with in vitro activated allogeneic CD4+ T cells. In addition, A20TGL leukemia/lymphoma cells (1.0×10⁶) were injected to these recipients 2 h before transplantation to induce leukemia. For FIGS. 4A and 4B, the numbers of DLL4^{hi}DC-induced CD4⁺ T cells were titrated from 0.5 million (M) to 2.5 M. B6 TCD-BM with or without addition of unstimulated B6 CD4⁺ TN were transferred to lethally irradiated BALB/c mice as controls. FIG. 4A depicts survival of the recipients over time. FIG. 4B depicts pictures showing in vivo detection of luciferase activity at day 14, 28 and 77 after transplantation. Data shown here are pooled from two independent experiments. FIG. 4C depicts histograms showing the expression of tested markers on the surface of DLL4^{hi}DC-induced CD4⁺ T cells. For FIGS. 4D through 4F, DLL4^{hi}DC-induced CD4⁺ T cells were flow-sorted into CD4⁺ CD44^{hi}CD62L^{lo} cells (0.5×10⁶) and CD4⁺ CD44^{lo}CD62L^{hi} cells (0.5×10⁶), and transferred together with TCD-BM into lethally irradiated leukemia BALB/c mice. FIG. 4D depicts survival of the recipients over time. FIG. 4E depicts a histogram showing GVHD clinical scores at day 10 and 20. FIG. 4F depicts in vivo images of the luciferase positive leukemia cells at day 14, 28, and 70 were shown. Photographs were taken by Xenogen IVIS 100. Data shown here are pooled from two independent experiments. *: P<0.05, **: p<0.01. ***: p<0.001.

FIG. 5, comprising FIG. 5A through 5G, depicts experimental results demonstrating DLL4^{hi}DC-T cells produce high levels of IFN-γ but have impaired capacity to expand in vivo. DLL4^{hi}DC-induced CD4⁺ T cells (1.0×10⁶) of B6/SJL origin (H-2^{b}, CD45.1) were transplanted with TCD-BM (5.0×10⁶) into lethally irradiated BALB/c mice (H-2^{d}, CD45.2). Equal numbers of unstimulated B6/SJL CD4+ TN were transferred to lethally irradiated BALB/c mice as controls. At day 3, 6, and 12 after transplantation, donor T cells were isolated from the spleens, lymph nodes (LN), liver, intestinal epithelial lymphocytes (IEL), and lamina propria lymphocytes (LPL) of recipient mice (three mice per group). FIG. 5A depicts graphs showing the numbers of donor (CD45.1⁺) CD4 T cells recovered from the spleens of recipient BALB/c mice at the indicated time points. FIG. 5B depicts a histogram showing Ki67 and CFSE in donor CD4⁺ T cells 6 days after in vivo transfer.

FIG. 5C depicts plots and graphs showing the percentage of early apoptotic donor CD4⁺ T cells that were Annexin V positive. FIG. 5D depicts graphs showing the percentages and numbers of donor CD4+ T cells producing IL-2 in the spleen. FIG. 5E depicts plots showing the percentage of donor FoxP3 positive regulatory T cells prior to transfer and 6 days after transfer. FIG. 5F depicts dot plots and graphs showing the percentages and numbers of donor CD4⁺ T cells producing IFN-γ, TNF-α, and IL-17 in the spleen at day 6 and 12 after transplantation. FIG. 5G depicts graphs showing the number of donor CD4⁺ T cells producing IFN-γ, TNF-α, and IL-17 in GVHD target organs at day 12 after transplantation. Data show mean±SD. Representative data from two independent experiments are shown. *: P<0.05, **: p<0.01. ***: p<0.001.

FIG. 6, comprising FIG. 6A through 6F, depicts experimental results demonstrating T cell IFN-γ is required for DLL4^{hi}DC programming to reduce CD4⁺ T cell mediated GVHD. Unstimulated WT or Ifng^{-/-} CD4⁺ TN (0.5×10⁶), and allogeneic DLL4^{hi}DC-induced WT or Ifng^{-/-}CD4⁺ T cells (0.5×10⁶) of B6 background were separately transplanted with TCD-BM (5.0×10⁶) into lethally irradiated BALB/c mice. FIG. 6A depicts survival of the recipients over time. FIG. 6B depicts the GVHD clinical score of the recipients over time. Data shown here are pooled from two independent experiments. For FIG. 6C, six days after transplantation, donor T cells were isolated from the spleens and livers. FIG. 6C depicts graphs showing the numbers of donor CD4⁺ T cells in recipient BALB/c mice (mean±SD). FIG. 6D depicts a graph showing the percentage of Annexin V⁺ cells among donor CD4⁺ T cells. FIG. 6E depicts plots and graphs showing the percentages of donor CD4⁺ T cells producing IFN-γ, IL-17, and TNF-α in the spleen and the liver. FIG. 6F depicts histograms showing the expression of indicated markers on the surface of unstimulated WT or Ifng^{-/-} CD4⁺ TN and DLL4^{hi}DC-stimulated WT or Ifng^{-/-} CD4⁺ T cells 7 days after in vivo transfer. Representative results of two independent experiments are shown. *: P<0.05. **: p<0.01. ***: p<0.001.

FIG. 7, comprising FIGS. 7A through 7D, depicts experimental results demonstrating DLL4 activation of Notch signaling is critical for DLL4^{hi}DCs to induce IFN-γ production in CD4⁺ T cells. BALB/c DLL4^{hi}DCs were cultured for five days with B6 WT CD4⁺ TN or DNMAML CD4⁺ TN. FIG. 7A depicts dot plots and graphs showing the percentages of IFN-γ- and IL17-producing cells cultured with or without addition of anti-DLL4 Ab. FIG. 7B depicts histograms and graphs showing the percentages and MFI of cleaved ICN1 in WT CD4⁺ T cells 72 hours after DLL4^{hi} DC activation, with or without addition of neutralizing anti-DLL4 Ab. FIG. 7C depicts graphs showing the relative expression of Notch target genes in WT CD4⁺ T cells after 5 days in culture. FIG. 7D depicts graphs showing the relative expression of indicated genes in WT CD4⁺ T cells after 5 days in culture. Representative data (mean±SD of triplicates) from two independent experiments are shown. *: P<0.05, **: p<0.01. ***: p<0.001.

FIG. 8 depicts the mRNA (SEQ ID NO:1) and amino acid (SEQ ID NO:2) sequences of human delta-like 4 (DLL4). The mRNA sequence of SEQ ID NO:1 was used for construction of a lentiviral vector for genetic engineering of DC and CART cells.

FIG. 9, comprising FIGS. 9A through 9B, depicts production of DLL4hiDCs from Mo-DCs transduced by lentivirus encoding DLL4. FIG. 9A depicts flow cytometry results from engineered monocyte derived DCs (DLL4 Mo-DCs) stained for HLA-DR, CD86, CD40 and DLL4. FIG. 9B depicts results demonstrating that DLL4^{hi} Mo-DCs have greater ability than Mo-DCs to induce TH1 cells.

FIG. 10, comprising FIGS. 10A through 10B, depicts DLL4-engineered chimeric antigen receptor (CAR) T cells specific for CD19 (CART-CD19). FIG. 10A depicts flow cytometry results showing a substantial proportion of CART-CD19 cells expressed high levels of DLL4, allowing these T cells expressing DLL4 to regulate T cell immune response. FIG. 10B depicts flow cytometry results demonstrating that introduction of DLL4 also induced TH17 cells.

FIG. 11 depicts the results of exemplary experiments demonstrating the generation of T cells expressing DLL4.

FIG. 12, comprising FIG. 12A through FIG. 12C, depicts the results of exemplary experiments demonstrating the efficacy of DLL4^{hi} DC-based vaccination in the induction and expansion of antigen-specific CD4 T cells and CD4 T cells producing high levels of IFN-γ compared to BM DCs produced from GM-CSF-based cultures (GM-DCs). FIG. 12A depicts exemplary flow cytometry analyses demonstrating that vaccination with murine DLL4+ DCs generated from bone marrow, pulsed with OVA for 3 hours resulted in a higher level of CD4 T cells and CD4 T cells producing high levels of IFN-γ. FIG. 12B depicts exemplary results of a quantification of the levels of CD4 T cells. FIG. 12C depicts exemplary results of a quantification of the levels of IFN-γ producing CD4 T cells.

### DETAILED DESCRIPTION

The present invention is based on the unexpected results that DLL4-DC-induced T cells provide a superior immunotherapeutic composition as they have a reduced risk of associated graft versus host disease toxicity. The invention provides compositions and methods of producing a population of immune cells useful in generating an immune response, and methods of use of the compositions of the invention as a vaccine in the treatment of cancer.

The present invention relates to methods and use of immune cells which express DLL4 (DLL4-IC) to induce clinically effective immune responses. In one embodiment, the DLL4-IC of the invention is a BM derived DC matured in a manner to activate DLL4 expression. In one embodiment, the DLL4-IC of the invention is a DC genetically engineered to express DLL4. In one embodiment, a DLL4-expressing DC (DLL4-DC) of the invention is a DLL4-expressing antigen presenting cell (DLL4-APC). However, the invention should not be limited to antigen presenting cells (APCs), rather the invention includes any immune cell cultured or genetically modified to express DLL4. None limiting examples of immune cells include but are not limited to B cells, T cells, NK cells, Macrophages, Dendritic cells, hematopoietic stem/progenitor cells and the likes.

In one embodiment, the DLL4-IC of the invention is a T-cell genetically engineered to express DLL4. In one embodiment, a T-cell genetically engineered to express DLL4 further comprises a chimeric antigen receptor (CAR).

The present invention further relates to methods and use of T cells having been induced in the presence of a population of DLL4-DCs to induce clinically effective immune responses. Therefore, the DLL4-IC of the invention includes a population of T cells, wherein the T cells have been induced by a DLL4-DC or a DLL4-APC. In one embodiment, the invention provides a method of in vitro stimulation of T cells by a population of DLL4-DCs from BM, whereby the T cells became alloreactive effector T cells that produced high levels of IFN-γ and IL-17. In one embodiment, DLL4-DC-induced T cells continually produce high levels of effector cytokines such as IFN-γ and TNF-αin vivo after transfer. In one embodiment, DLL4-DC-induced T cells elicit anti-tumor activity with a wider safety range of infused T cell numbers without causing GVHD.

In one embodiment, the DLL4-DC of the present invention have the capacity to condition toward strong Th1 cellular responses. In one embodiment, the immune cells of the present invention have the capacity to condition toward strong Th17 cellular responses.

In one embodiment, the invention provides a method of providing anti-tumor immunity in a subject, the method comprising: administering to the subject an effective amount of a DLL4-IC of the invention. The DLL4-ICs of the invention have the ability to induce clinically effective immune responses. The DLL4-ICs of the present invention further produce desirable levels of cytokines and chemokines, and have the capacity to induce apoptosis of tumor cells.

In one embodiment, the invention relates to an improved vaccine for treatment of a cancer. In one embodiment, the invention relates to method of adoptive transfer of DLL4-IC into a patient for treatment of a cancer. In one embodiment, a cancer is a B-cell malignancy. In one embodiment, a cancer is leukemia. In one embodiment, the invention provides a population of DLL4-DCs loaded with leukemia-associated antigens for use in selection and expansion of leukemic cell-reactive, DLL4-DC-induced T cells to mediate anti-leukemia activity.

The invention includes a method of generating DLL4-IC for use in immunotherapy. In one embodiment, the method comprises activating immature BM-DC with Flt3L and one or more TLR agonist. In one embodiment, the one or more TLR agonist activates one or more of TLR7, TLR8 and TLR4. In one embodiment, the at least one TLR agonist is one or more of LPS, R848, imiquimod, monophosphoryl lipid A, and VTX-2337. In one embodiment, the method comprises activating immature BM-DC with Flt3L, LPS and R848.

In one embodiment, the method comprises activating antigen loaded BM-DC with Flt3L and one or more TLR agonist. In one embodiment, the antigen is a tumor antigen. In another embodiment, the antigen is a microbial antigen. In yet another embodiment, the one or more TLR agonist is LPS and R848. In yet another embodiment, the DC exhibits a killer function whereby the DC are capable of lysing targeted cancer cells.

The invention includes a method of engineering a population of DLL4-IC for use in immunotherapy. In one embodiment, the method comprises viral introduction of DLL4 into monocyte-derived DCs. In one embodiment, the method comprises viral introduction of DLL4 into a T cell. In one embodiment, the invention provides a T cell genetically engineered to express both DLL4 and a CAR. In one embodiment, the genetically engineered T cell of the invention is specific for CD19. However, the invention should not be limited to CD19 specific CAR T cells. Rather, the invention include genetically modifying any CAR T cells to express DLL4.

In one embodiment, the genetically modified immune cells of the invention stably express DLL4. In one embodiment, the genetically modified immune cells of the invention comprise a nucleic acid sequence encoding DLL4. In one embodiment, a nucleic acid sequence is an RNA sequence and is encoded on a lentiviral vector.

In one embodiment, the DLL4-IC of the invention is generated by introducing a lentiviral vector comprising a nucleic acid sequence of DLL4 (SEQ ID NO:1). In one embodiment, a lentiviral vector comprising the mRNA sequence of DLL4 (SEQ ID NO:1) is introduced into a T cell. In one embodiment, a lentiviral vector comprising the mRNA sequence of DLL4 (SEQ ID NO:1) is introduced into a DC. In one embodiment, a lentiviral vector comprising the mRNA sequence of DLL4 (SEQ ID NO:1) is introduced into a T cell. In one embodiment, a lentiviral vector comprising the mRNA sequence of DLL4 (SEQ ID NO:1) is introduced into a CAR T cell. In one embodiment, a lentiviral vector comprising the mRNA sequence of DLL4 (SEQ ID NO:1) is introduced into a NKT cell.

In one embodiment the invention relates to administering a DLL4-IC for the treatment of a patient having cancer or at risk of having cancer. In one embodiment, DLL4-IC are used in the treatment of an advanced B cell malignancy. In one embodiment, autologous BM cells are collected from a patient in need of treatment and DC are activated and expanded using the methods described herein and known in the art and then infused back into the patient. In one embodiment, autologous PBMCs are collected from a patient in need of treatment and DC are activated, genetically engineered and expanded using the methods described herein and known in the art and then infused back into the patient. In one embodiment, autologous PBMCs are collected from a patient in need of treatment and T cells are activated, genetically engineered and expanded using the methods described herein and known in the art and then infused back into the patient.

The invention further includes a method of eliciting an immune response in a mammal, the method comprises administering a composition comprising a population of DLL4-IC to the mammal in need thereof. In one embodiment, the immunogenic population exhibits a killer function whereby the cells are capable of lysing targeted cancer cells.

In one embodiment, the invention provides DLL4 protein-conjugated magnetic beads, which can be used for regulating T cell expansion and function. For example, DLL4 protein-conjugated magnetic beads can be used together with anti-CD3-Ab and anti-CD28 Ab to expand T cells in cultures for the purpose of adoptive T-cell therapy for cancer, hematopoietic stem cell transplantation and autoimmune diseases.

In one embodiment, the DLL4-protein-conjugated protein complex can be used as an immune adjuvant.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, exemplary materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e. to at least one) of the grammatical object of the article. By way of example, “an element” means one element or more than one element.

“About” as used herein when referring to a measurable value such as an amount, a temporal duration, and the like, is meant to encompass non-limiting variations of ±40% or ±20% or ±10%, ±5%, ±1%, or ±0.1% from the specified value, as such variations are appropriate.

“Allogeneic” refers to a graft derived from a different animal of the same species.

As used herein, the term “autologous” is meant to refer to any material derived from the same individual to which it is later to be re-introduced into the individual.

The term “antibody” as used herein, refers to an immunoglobulin molecule, which is able to specifically bind to a specific epitope on an antigen. Antibodies can be intact immunoglobulins derived from natural sources or from recombinant sources and can be immunoactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1988; Houston et al., 1988; Bird et al., 1988).

The term “antigen” or “ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The term “anti-tumor effect” as used herein, refers to a biological effect which can be manifested by a decrease in tumor volume, a decrease in the number of tumor cells, a decrease in the number of metastases, an increase in life expectancy, or amelioration of various physiological symptoms associated with the cancerous condition. An “anti-tumor effect” can also be manifested by the ability of the peptides, polynucleotides, cells and antibodies of the invention in prevention of the occurrence of tumor in the first place.

“An antigen presenting cell” (APC) is a cell that are capable of activating T cells, and includes, but is not limited to, monocytes/macrophages, B cells and dendritic cells (DCs).

The term “dendritic cell” or “DC” refers to any member of a diverse population of morphologically similar cell types found in lymphoid or non-lymphoid tissues. These cells are characterized by their distinctive morphology and high levels of surface MHC-class II expression. DCs can be isolated from a number of tissue sources. DCs have a high capacity for sensitizing MHC-restricted T cells and are very effective at presenting antigens to T cells in situ. The antigens may be self-antigens that are expressed during T cell development and tolerance, and foreign antigens that are present during normal immune processes.

As used herein, an “activated DC” is a DC that has been exposed to a Toll-like receptor agonist. The activated DC may or may not be loaded with an antigen.

As used herein, an “activated T cell” refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

The term “mature DC” as used herein, is defined as a dendritic cell that expresses molecules, including high levels of MHC class II, CD80 (B7.1) and CD86 (B7.2). In contrast, immature dendritic cells express low levels of MHC class II, CD80 (B7.1) and CD86 (B7.2) molecules, yet can still take up an antigen.

“Antigen-loaded APC” or an “antigen-pulsed APC” includes an APC, which has been exposed to an antigen and activated by the antigen. For example, an APC may become Ag-loaded in vitro, e.g., during culture in the presence of an antigen. The APC may also be loaded in vivo by exposure to an antigen. An “antigen-loaded APC” is traditionally prepared in one of two ways: (1) small peptide fragments, known as antigenic peptides, are “pulsed” directly onto the outside of the APCs; or (2) the APC is incubated with whole proteins or protein particles which are then ingested by the APC. These proteins are digested into small peptide fragments by the APC and are eventually transported to and presented on the APC surface. In addition, the antigen-loaded APC can also be generated by introducing a polynucleotide encoding an antigen into the cell.

The term “autoimmune disease” as used herein is defined as a disorder that results from an autoimmune response. An autoimmune disease is the result of an inappropriate and excessive response to a self-antigen. Examples of autoimmune diseases include but are not limited to, Addision's disease, alopecia areata, ankylosing spondylitis, autoimmune hepatitis, autoimmune parotitis, Crohn's disease, diabetes (Type I), dystrophic epidermolysis bullosa, epididymitis, glomerulonephritis, Graves' disease, Guillain-Barr syndrome, Hashimoto's disease, hemolytic anemia, systemic lupus erythematosus, multiple sclerosis, myasthenia gravis, pemphigus vulgaris, psoriasis, rheumatic fever, rheumatoid arthritis, sarcoidosis, scleroderma, Sjogren's syndrome, spondyloarthropathies, thyroiditis, vasculitis, vitiligo, myxedema, pernicious anemia, ulcerative colitis, among others.

As used herein, the term “autologous” is meant to refer to any material derived from the same individual to which it is later to be re-introduced into the individual.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, brain cancer, bladder cancer, breast cancer, cervical cancer, colorectal cancer, liver cancer, kidney cancer, lymphoma, leukemia, lung cancer, melanoma, metastatic melanoma, mesothelioma, neuroblastoma, ovarian cancer, prostate cancer, pancreatic cancer, renal cancer, skin cancer, thymoma, sarcoma, non-Hodgkin's lymphoma, Hodgkin's lymphoma, uterine cancer, and the like.

“Donor antigen” refers to an antigen expressed by the donor tissue to be transplanted into the recipient.

“Recipient antigen” refers to a target for the immune response to the donor antigen.

As used herein, an “effector cell” refers to a cell which mediates an immune response against an antigen. An example of an effector cell includes, but is not limited to, a T cell and a B cell.

As used herein, “endogenous” refers to any material from or produced inside an organism, cell, tissue or system.

As used herein, the term “exogenous” refers to any material introduced from or produced outside an organism, cell, tissue or system.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “epitope” as used herein is defined as a small chemical molecule on an antigen that can elicit an immune response, inducing B and/or T cell responses. An antigen can have one or more epitopes. Most antigens have many epitopes; i.e., they are multivalent. In general, an epitope is roughly five amino acids and/or sugars in size. One skilled in the art understands that generally the overall three-dimensional structure, rather than the specific linear sequence of the molecule, is the main criterion of antigenic specificity and therefore distinguishes one epitope from another.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

The term “helper T cell” as used herein is defined as an effector T cell whose primary function is to promote the activation and functions of other B and T lymphocytes and or macrophages. Most helper T cells are CD4 T cells.

As used herein, “immunogen” refers to a substance that is able to stimulate or induce a humoral antibody and/or cell-mediated immune response in a mammal.

The term “immunoglobulin” or “Ig”, as used herein is defined as a class of proteins, which function as antibodies. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most mammals. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

As used herein, “in vitro transcribed RNA” refers to RNA, e.g., mRNA, that has been synthesized in vitro. Generally, the in vitro transcribed RNA is generated from an in vitro transcription vector. The in vitro transcription vector comprises a template that is used to generate the in vitro transcribed RNA.

The term “major histocompatibility complex”, or “MHC”, as used herein is defined as a specific cluster of genes, many of which encode evolutionarily related cell surface proteins involved in antigen presentation, which are among the most important determinants of histocompatibility. Class I MHC, or MHC-I, function mainly in antigen presentation to CD8 T lymphocytes. Class II MHC, or MHC-II, function mainly in antigen presentation to CD4 T lymphocytes.

As used herein, the term “modulate” is meant to refer to any change in biological state, i.e. increasing, decreasing, and the like.

The term “polypeptide” as used herein is defined as a chain of amino acid residues, usually having a defined sequence. As used herein the term polypeptide is mutually inclusive of the terms “peptide” and “protein”.

“Proliferation” is used herein to refer to the reproduction or multiplication of similar forms of entities, for example proliferation of a cell. That is, proliferation encompasses production of a greater number of cells, and can be measured by, among other things, simply counting the numbers of cells, measuring incorporation of ³H-thymidine into the cell, and the like.

The term “self-antigen” as used herein is defined as an antigen that is expressed by a host cell or tissue. Self-antigens may be tumor antigens, but in certain embodiments, are expressed in both normal and tumor cells. A skilled artisan would readily understand that a self-antigen may be overexpressed in a cell.

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are culture in vitro. In other embodiments, the cells are not cultured in vitro.

The term “T cell” as used herein is defined as a thymus-derived cell that participates in a variety of cell-mediated immune reactions.

The term “B cell” as used herein is defined as a cell derived from the bone marrow and/or spleen. B cells can develop into plasma cells which produce antibodies.

The term “Toll like receptor”, or “TLR” as used herein is defined as a class of proteins that play a role in the innate immune system. TLRs are single membrane-spanning, non-catalytic receptors that recognize structurally conserved molecules derived from microbes. TLRs activate immune cell responses upon binding to a ligand.

The term “Toll like receptor agonists”, or “TLR agonists” as used herein is defined as a ligand that binds to the TLR to activate immune cell response.

As used herein, a “therapeutically effective amount” is the amount of a therapeutic composition sufficient to provide a beneficial effect to a mammal to which the composition is administered.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “vaccine” as used herein is defined as a material used to provoke an immune response after administration of the material to a subject, e.g., a human or a non-human animal.

“Xenogeneic” refers to a graft derived from an animal of a different species.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The invention provides compositions and methods of producing a population of immune cells useful in generating an immune response, and methods of use of the compositions of the invention as a vaccine in the treatment of cancer.

The present invention relates to methods and use of immune cells which express DLL4 (DLL4-IC) to induce clinically effective immune responses. In one embodiment, the DLL4-IC of the invention is a BM derived DC matured in a manner to activate DLL4 expression. In one embodiment, the DLL4-IC of the invention is a DC genetically engineered to express DLL4. In one embodiment, a DLL4-expressing DC (DLL4-DC) of the invention is a DLL4-expressing antigen presenting cell (DLL4-APC). However, the invention should not be limited to antigen presenting cells. Rather, the invention includes any immune cell cultured or genetically modified to express DLL4. None limiting examples of immune cells include but are not limited to B cells, T cells, NK cells, Macrophages, Dendritic cells, and the likes.

In one embodiment, the invention provides DLL4 protein-conjugated magnetic beads, which can be used for regulating T cell expansion and function. For example, DLL4 protein-conjugated magnetic beads can be used together with anti-CD3-Ab and anti-CD28 Ab to expand T cells in cultures for the purpose of adoptive T-cell therapy for cancer, hematopoietic stem cell transplantation and autoimmune diseases.

In one embodiment, the DLL4-protein-conjugated protein complex can be used as an immune adjuvant.

### Generating DLL4-DCs

One aspect of the invention is the generation of DLL4-DCs from bone marrow cells. Methods appropriate for isolating and optionally purifying bone marrow cells for use in the methods of the invention are generally known to those of skill in the art. The dendritic cells are obtained from the bone marrow cells by culturing the latter in medium containing Flt3L and at least one TLR agonist. In one embodiment, the dendritic cells are obtained from the bone marrow cells by culturing the latter in medium containing Flt3L, LPS and R848 (resiquimod). Flt3L, LPS and R848 suitable for use in the invention are commercially available. In one embodiment, Flt3L is used at a concentration in the range of 10 to 100 ng/mL. In one embodiment, Flt3L is used at a concentration of 50 ng/mL. In one embodiment, LPS and R848 are used at saturating concentration. That is, they are used at a concentration in which all the TLR4 and TLR7/8 receptors on the bone marrow cells are occupied by the biologically active LPS and R848 molecules. Of course, the actual concentration may depend on the quality or lot of LPS and R848 used. In one embodiment, saturating concentration for LPS or R848 was 100 ng/mL. The cells can be co-cultured in standard tissue culture medium with standard additives, such as RPMI 1640 supplemented with 10% fetal bovine serum, 10 mM Hepes, 2 mM L-glutamine, 5×10⁻⁵ M 2-mercaptoethanol, penicillin (100 U/mL) and streptomycin (100 mg/μL). In one embodiment, the bone marrow cells are cultured in the presence of the cytokines for between 8 to 12 days. In one embodiment, the cells are cultured in the presence of the cytokines for 9 days.

In one embodiment, the method of the invention comprises the following steps: (1) providing a sample of naïve T cells; (2) providing a sample of dendritic cells produced by the process comprising the steps of (a) treating bone marrow-derived dendritic cells with Flt3L and at least one TLR agonist, and (b) isolating treated bone marrow dendritic cells that express DLL4; (3) co-culturing the naïve T cells and the DLL4-DC cells; and (4) isolating the DLL4-DC stimulated T cells. In one embodiment, the method of producing the dendritic cells further includes the step of (c) activating the dendritic cell with at least one antigen. In one embodiment, the method further comprises step (5) administering the DLL4-DC stimulated T cells to a patient in need thereof.

In one embodiment, the method of the invention comprises the following steps: (1) providing a sample of naïve T cells; (2) providing a sample of dendritic cells produced by the process comprising the steps of (a) generating monocyte derived-DCs from PBMC, (b) genetically modifying the monocytes to express DLL4 and (c) maturing the dendritic cells; (3) co-culturing the naïve T cells and the DLL4-DC cells; and (4) isolating the DLL4-DC stimulated T cells. In one embodiment, the method of producing the dendritic cells further includes the step of (d) activating the dendritic cell with at least one antigen. In one embodiment, the method further comprises administering the DLL4-DC stimulated T cells to a patient in need thereof.

In one embodiment, the DCs of the invention are derived from cells obtained from the same individual from whom the naïve T cells are taken. In one embodiment, the sample of naïve T cells consist of CD4⁺ T cells from the peripheral blood of a patient who is to be the recipient of a treatment or transplant. Obtaining T cell populations employs techniques well known in the art which are fully described in DiSabato et al, eds., Meth. in Enzymol., Vol. 108 (1984). For example, the CD4⁺ T cells can be isolated as follows: first mononuclear cells are isolated from the peripheral blood and depleted of adherent cells; CD4⁺ T cells are then purified by depleting other cell types, e.g. by immunomagnetic depletion (e.g. with Dynabeads, Dynal, Oslo, Norway), or the like, using a cocktail of commercially available monoclonal antibodies, e.g. anti-CD14, anti-CD16, anti-CD20, anti-CD8, anti-CD40 (available from Becton-Dickinson and/or Ortho Diagnostic Systems, New Jersey). CD4⁺ populations having greater than 95% purity are typically achieved after two rounds of immunomagnetic depletion.

In one embodiment, the DCs of the invention are derived from cells obtained from a different individual than that from whom the naïve T cells are taken; that is, the stimulator cells are allogenic with respect to the naïve T cells. These cells are obtained as described above.

### Maturation of DCs

Both BM-derived and genetically engineered DCs of the invention require maturation signals to enhance their antigen-presenting capacity. In one embodiment, a mixture of cytokines, including TNF-α, IL-1β, IL-6 and prostaglandin E2 (PGE2), have the ability to mature genetically engineered DCs of the invention. In one embodiment, one or more TLR agonists are used to mature genetically engineered DCs. Genetically engineered DCs of the invention can also be matured with calcium ionophore prior to being pulsed with antigen. In one embodiment, genetically engineered DCs are matured with Flt3L and one or more TLR agonist.

In one embodiment, BM-derived DCs are matured with Flt3L and one or more TLR agonist. This approach specially matures the dendritic cells in a manner to stimulate expression of DLL4. This unique activation method endows the DCs with qualities not found in BM-DCs that are matured using other methods (e.g., using a combination of GM-CSF and TLR agonists).

In one embodiment, the BM-DCs of the present invention can be activated with the combination of Flt3L and the TLR4 agonist, bacterial lipopolysaccharide (LPS) and the TLR7/8 agonist resimiquod (R848). In other embodiments, TLR2 agonists, such as lipotechoic acid (LTA), TLR3 agonists, such as poly(I:C), and/or other TLR4 agonists, such as MPL, may be used. As contemplated herein, any TLR agonist, or combination of TLR agonists, can be used to active DCs, provided such ligands stimulate the production of cytokine and chemokine signals by the activated DCs. Many other TLR agonists are known in the art and can be found in the published literature for use with the present invention. In one embodiment, the BM-DCs of the present invention can be activated with the combination of Flt3L, one or more TLR agonists and INF-γ.

### DC-Production of Signal

Mature DCs have the ability to activate a T cell-mediated immune responses (Jonuleit et al., 2001, Int. J. Cancer. 93:243-251; Prabakaran et al., 2002, Ann. Surg. Oncol. 9:411-418; Xu et al., 2003, J. Immunol. 171:2251-2261), in part because specific cytokines are secreted by mature DCs, which potentiate a T cell response.

The cytokines produced by mature DCs have varied effects on T cell response. For example, IL-12, a heterodimeric cytokine, is produced by DCs, and is pivotal in producing IFN-γ-secreting CD4⁺ and CD8⁺ T cells and enhancing antibacterial and anti-tumor responses (Gee et al., 2009, Inflamm. Allergy Drug Targets 8:40-52). IL-12 can also inhibit the growth of primary tumor as well as metastatic tumor cells in ovarian carcinoma (OV-HM) murine models (Tatsumi et al., 2001, Cancer Res. 61:7563-7567). IL-12 can also mediate the generation of high-avidity anti-tumor T cells (Xu et al., 2003, J. Immunol. 171:2251-2261), thereby improving anti-tumor T cell function. DCs also produce chemokines as a fourth signal that leads to the accumulation of T cells and further effects T cell responses (Xiao et al., 2003, Cytokine 23:126-132).

DCs can secrete other cytokines that further influence T cell activation. For example, DCs can secrete IL-1, IL-6 and IL-23, which activate Th17 cells. Th17 cells are a recently defined subset of proinflammatory T cells that contribute to pathogen clearance and tissue inflammation by means of the production of their signature cytokine, IL-17 (Kikly et al., 2006, Curr. Opin. Immunol. 18:670-675). IL-12 production can lead to a more potent Th1 response, whereas IL-23 production can lead to the maturation of Th17 cells. In fact, IL-12 producing DCs can polarize a predominantly Th1 response in the presence of IL-23, yet by contrast, DCs that produce IL-23 in the absence of IL-12 polarize a strong Th17 response (Roses et al., 2008, J. Immunol. 181:5120-5127; Acosta-Rodriguez et al., 2007, Nat. Immunol. 8:639-646). Thus, specific DC-secreted cytokines have a profound impact on T-cell function, highlighting the importance of delineating the cytokine profile of mature DCs.

In one embodiment, DLL4-DC of the invention induce a TH1 cell response. In one embodiment, DLL4-DC of the invention activate T cells that produce high levels of IFN-γ. In one embodiment, DLL4-DC of the invention induce a TH17 cell response. In one embodiment, the DLL4-DC of the invention activate T cells that produce high levels of IFN-γ and IL-17. In one embodiment, the DLL4-DC of the invention activate T cells that produce high levels of TNF-α and IL-17. In one embodiment, the DLL4-DC of the invention activate T cells that produce IL-4, TNF-α, and IL-17.

### Generation of a Loaded (Pulsed) Immune Cell

The present invention includes a cell that has been exposed or otherwise “pulsed” with an antigen. For example, an APC, such as a DC, may become Ag-loaded in vitro, e.g., by culture ex vivo in the presence of an antigen, or in vivo by exposure to an antigen.

A person skilled in the art would also readily understand that an APC can be “pulsed” in a manner that exposes the APC to an antigen for a time sufficient to promote presentation of that antigen on the surface of the APC. For example, an APC can be exposed to an antigen in the form of small peptide fragments, known as antigenic peptides, which are “pulsed” directly onto the outside of the APCs; or APCs can be incubated with whole proteins or protein particles which are then ingested by the APCs. These whole proteins are digested into small peptide fragments by the APC and eventually carried to and presented on the APC surface. Antigen in peptide form may be exposed to the cell by standard “pulsing” techniques described herein.

Without wishing to be bound by any particular theory, the antigen in the form of a foreign or an autoantigen is processed by the APC of the invention in order to retain the immunogenic form of the antigen. The immunogenic form of the antigen implies processing of the antigen through fragmentation to produce a form of the antigen that can be recognized by and stimulate immune cells, for example T cells. In one embodiment, such a foreign or an autoantigen is a protein which is processed into a peptide by the APC. The relevant peptide which is produced by the APC may be extracted and purified for use as an immunogenic composition. Peptides processed by the APC may also be used to induce tolerance to the proteins processed by the APC.

The antigen-loaded APC, otherwise known as a “pulsed APC” of the invention, is produced by exposure of the APC to an antigen either in vitro or in vivo. In the case where the APC is pulsed in vitro, the APC can be plated on a culture dish and exposed to an antigen in a sufficient amount and for a sufficient period of time to allow the antigen to bind to the APC. The amount and time necessary to achieve binding of the antigen to the APC may be determined by using methods known in the art or otherwise disclosed herein. Other methods known to those of skill in the art, for example immunoassays or binding assays, may be used to detect the presence of antigen on the APC following exposure to the antigen.

In a further embodiment of the invention, the APC may be transfected with a vector which allows for the expression of a specific protein by the APC. The protein which is expressed by the APC may then be processed and presented on the cell surface on an MHC receptor. The transfected APC may then be used as an immunogenic composition to produce an immune response to the protein encoded by the vector.

As discussed elsewhere herein, vectors may be prepared to include a specific polynucleotide which encodes and expresses a protein to which an immunogenic response is desired. In one embodiment, retroviral vectors are used to infect the cells. In one embodiment, adenoviral vectors are used to infect the cells.

In another embodiment, a vector may be targeted to an APC by modifying the viral vector to encode a protein or portions thereof that is recognized by a receptor on the APC, whereby occupation of the APC receptor by the vector will initiate endocytosis of the vector, allowing for processing and presentation of the antigen encoded by the nucleic acid of the viral vector. The nucleic acid which is delivered by the virus may be native to the virus, which when expressed on the APC encodes viral proteins which are then processed and presented on the MHC receptor of the APC.

As contemplated herein, various methods can be used for transfecting a polynucleotide into a host cell. The methods include, but are not limited to, calcium phosphate precipitation, lipofection, particle bombardment, microinjection, electroporation, colloidal dispersion systems (i.e. macromolecule complexes, nanocapsules, microspheres, beads, and lipid-based systems including oil-in-water emulsions, micelles, mixed micelles, and liposomes). These methods are understood in the art and are described in published literature so as to enable one skilled in the art to perform these methods.

In another embodiment, a polynucleotide encoding an antigen can be cloned into an expression vector and the vector can be introduced into an APC to otherwise generate a loaded APC. Various types of vectors and methods of introducing nucleic acids into a cell are discussed in the available published literature. For example, the expression vector can be transferred into a host cell by physical, chemical or biological means. See, for example, Sambrook et al. (2001, Molecular Cloning: A Laboratory Manual, Cold Spring Harbor Laboratory, New York), and in Ausubel et al. (1997, Current Protocols in Molecular Biology, John Wiley & Sons, New York). It is readily understood that the introduction of the expression vector comprising a polynucleotide encoding an antigen yields a pulsed cell.

The present invention includes various methods for pulsing APCs including, but not limited to, loading APCs with whole antigen in the form of a protein, cDNA or mRNA. However, the invention should not be construed to be limited to the specific form of the antigen used for pulsing the APC. Rather, the invention encompasses other methods known in the art for generating an antigen loaded APC. In one embodiment, the APC is transfected with mRNA encoding a defined antigen. mRNA corresponding to a gene product whose sequence is known can be rapidly generated in vitro using appropriate primers and reverse transcriptase-polymerase chain reaction (RT-PCR) coupled with transcription reactions. Transfection of an APC with an mRNA provides an advantage over other antigen-loading techniques for generating a pulsed APC. For example, the ability to amplify RNA from a microscopic amount of tissue, i.e. tumor tissue, extends the use of the APC for vaccination to a large number of patients.

There are many methods that can be used for engineering DCs and other APCs, such as mRNA-based delivering, DNA-plasmid-based delivering, all of which are encompassed in the invention. That is, any delivery system can be used to engineer immune cells to express DLL4.

For an antigenic composition to be useful as a vaccine, the antigenic composition must induce an immune response to the antigen in a cell, tissue or mammal (e.g., a human). As used herein, an “immunological composition” may comprise an antigen (e.g., a peptide or polypeptide), a nucleic acid encoding an antigen (e.g., an antigen expression vector), or a cell expressing or presenting an antigen or cellular component. In particular embodiments the antigenic composition comprises or encodes all or part of any antigen described herein, or an immunologically functional equivalent thereof. In other embodiments, the antigenic composition is in a mixture that comprises an additional immunostimulatory agent or nucleic acids encoding such an agent. Immunostimulatory agents include but are not limited to an additional antigen, an immunomodulator, an antigen presenting cell or an adjuvant. In other embodiments, one or more of the additional agent(s) is covalently bonded to the antigen or an immunostimulatory agent, in any combination. In certain embodiments, the antigenic composition is conjugated to or comprises an HLA anchor motif amino acids.

A vaccine, as contemplated herein, may vary in its composition of nucleic acid and/or cellular components. In a non-limiting example, a nucleic encoding an antigen might also be formulated with an adjuvant. Of course, it will be understood that various compositions described herein may further comprise additional components. For example, one or more vaccine components may be comprised in a lipid or liposome. In another non-limiting example, a vaccine may comprise one or more adjuvants. A vaccine of the present invention, and its various components, may be prepared and/or administered by any method disclosed herein or as would be known to one of ordinary skill in the art, in light of the present disclosure.

It is understood that an antigenic composition of the present invention may be made by a method that is well known in the art, including but not limited to chemical synthesis by solid phase synthesis and purification away from the other products of the chemical reactions by HPLC, or production by the expression of a nucleic acid sequence (e.g., a DNA sequence) encoding a peptide or polypeptide comprising an antigen of the present invention in an in vitro translation system or in a living cell. In addition, an antigenic composition can comprise a cellular component isolated from a biological sample. The antigenic composition isolated and extensively dialyzed to remove one or more undesired small molecular weight molecules and/or lyophilized for more ready formulation into a desired vehicle. It is further understood that additional amino acids, mutations, chemical modification and such like, if any, that are made in a vaccine component will not substantially interfere with the antibody recognition of the epitopic sequence.

A peptide or polypeptide corresponding to one or more antigenic determinants of the present invention should generally be at least five or six amino acid residues in length, and may contain up to about 10, about 15, about 20, about 25, about 30, about 35, about 40, about 45 or about 50 residues or so. A peptide sequence may be synthesized by methods known to those of ordinary skill in the art, such as, for example, peptide synthesis using automated peptide synthesis machines, such as those available from Applied Biosystems, Inc., Foster City, Calif. (Foster City, Calif.).

Longer peptides or polypeptides also may be prepared, e.g., by recombinant means. In certain embodiments, a nucleic acid encoding an antigenic composition and/or a component described herein may be used, for example, to produce an antigenic composition in vitro or in vivo for the various compositions and methods of the present invention. For example, in certain embodiments, a nucleic acid encoding an antigen is comprised in, for example, a vector in a recombinant cell. The nucleic acid may be expressed to produce a peptide or polypeptide comprising an antigenic sequence. The peptide or polypeptide may be secreted from the cell, or comprised as part of or within the cell.

In certain embodiments, an immune response may be promoted by transfecting or inoculating a mammal with a nucleic acid encoding an antigen. One or more cells comprised within a target mammal then expresses the sequences encoded by the nucleic acid after administration of the nucleic acid to the mammal. A vaccine may also be in the form, for example, of a nucleic acid (e.g., a cDNA or an RNA) encoding all or part of the peptide or polypeptide sequence of an antigen. Expression in vivo by the nucleic acid may be, for example, by a plasmid type vector, a viral vector, or a viral/plasmid construct vector.

In another embodiment, the nucleic acid comprises a coding region that encodes all or part of the sequences encoding an appropriate antigen, or an immunologically functional equivalent thereof. Of course, the nucleic acid may comprise and/or encode additional sequences, including but not limited to those comprising one or more immunomodulators or adjuvants.

### Antigens

As contemplated herein, the present invention may include use of any antigen suitable for loading into an APC to elicit an immune response. In one embodiment, an antigen is a tumor antigen. Tumor antigens can be divided into two broad categories: shared tumor antigens; and unique tumor antigens. Shared antigens are expressed by many tumors, while unique tumor antigens can result from mutations induced through physical or chemical carcinogens, and are therefore expressed only by individual tumors. In certain embodiments, shared tumor antigens are loaded into the DCs of the present invention. In other embodiments, unique tumor antigens are loaded into the DCs of the present invention.

In the context of the present invention, “tumor antigen” refer to antigens that are common to specific hyperproliferative disorders. In certain aspects, the hyperproliferative disorder antigens of the present invention are derived from cancers, including but not limited to, primary or metastatic melanoma, thymoma, lymphoma, sarcoma, lung cancer, liver cancer, non-Hodgkin's lymphoma, Hodgkins lymphoma, leukemias, uterine cancer, cervical cancer, bladder cancer, kidney cancer and adenocarcinomas such as breast cancer, prostate cancer, ovarian cancer, pancreatic cancer, and the like. In one embodiment, the hyperproliferative disorder is an advanced B cell malignancy.

The type of tumor antigen referred to in the invention may be a tumor-specific antigen (TSA) or a tumor-associated antigen (TAA). A TSA is unique to tumor cells and does not occur on other cells in the body. A TAA associated antigen is not unique to a tumor cell and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development when the immune system is immature and unable to respond or they may be antigens that are normally present at extremely low levels on normal cells but which are expressed at much higher levels on tumor cells.

Non-limiting examples of TSA or TAA antigens include the following: Differentiation antigens such as MART-1/MelanA (MART-I), gp100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG16, TA-90\Mac-2 binding protein\cyclophilin C-associated protein, TAAL6, TAG72, TLP, and TPS.

Malignant tumors express a number of proteins that can serve as target antigens for an immune attack. For example, in B cell lymphoma, the tumor-specific idiotype immunoglobulin constitutes a truly tumor-specific immunoglobulin antigen that is unique to the individual tumor. B cell differentiation antigens, such as CD19, CD20 and CD37, are other candidates for target antigens in B cell lymphoma. Some of these antigens (CEA, HER-2, CD19, CD20, idiotype) have been used as targets for passive immunotherapy with monoclonal antibodies with limited success.

The tumor antigen and the antigenic cancer epitopes thereof may be purified and isolated from natural sources such as from primary clinical isolates, cell lines and the like. The cancer peptides and their antigenic epitopes may also be obtained by chemical synthesis or by recombinant DNA techniques known in the arts. Techniques for chemical synthesis are described in Steward et al. (1969); Bodansky et al. (1976); Meienhofer (1983); and Schroder et al. (1965). Furthermore, as described in Renkvist et al. (2001), there are numerous antigens known in the art. Although analogs or artificially modified epitopes are not specifically described, a skilled artisan recognizes how to obtain or generate them by standard means in the art. Other antigens, identified by antibodies and as detected by the Serex technology (see Sahin et al. (1997) and Chen et al. (2000)), are identified in the database of the Ludwig Institute for Cancer Research.

In yet another embodiment, the present invention may include microbial antigens for presentation by the APCs. As contemplated herein, microbial antigens may be viral, bacterial, or fungal in origin. Examples of infectious virus include: Retroviridae (e.g. human immunodeficiency viruses, such as HIV-1 (also referred to as HTLV-III, LAV or HTLV-III/LAV, or HIV-III; and other isolates, such as HIV-LP; Picornaviridae (e.g. polio viruses, hepatitis A virus; enteroviruses, human coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronaviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairo viruses); Arena viridae (hemorrhagic fever viruses); Reoviridae (e.g. reoviruses, orbiviruses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus); Poxviridae (variola viruses, vaccinia viruses, pox viruses); and Iridoviridae (e.g. African swine fever virus); and unclassified viruses (e.g. the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses).

Examples of infectious bacteria include: Helicobacter pyloris, Borelia burgdorferi, Legionella pneumophilia, Mycobacteria sps (e.g. M. tuberculosis, M. avium, M. intracellulare, M. kansasii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus anthracis, corynebacterium diphtheriae, corynebacterium sp., Erysipelothrix rhusiopathiae, Clostridium perfringens, Clostridium tetani, Enterobacter aerogenes, Klebsiella pneumoniae, Pasturella multocida, Bacteroides sp., Fusobacterium nucleatum, Streptobacillus moniliformis, Treponema Treponema pertenue, Leptospira, and Actinomyces israelli.

Examples of infectious fungi include: Cryptococcus neoformans, Histoplasma capsulatum, Coccidioides immitis, Blastomyces dermatitidis, Chlamydia trachomatis, Candida albicans. Other infectious organisms (i.e., protists) including: Plasmodium falciparum and Toxoplasma gondii.

### Generating Immune Cells which Express DLL4 (DLL4-IC)

In one embodiment, an immunogenic composition of the invention is a population of immune cells that express DLL4. Any immune cell is encompassed in the invention. In addition, there are many methods that can be used for engineering cells, such as mRNA-based delivering, DNA-plasmid-based delivering, all of which are encompassed in the invention. That is, any delivery system can be used to engineer immune cells to express DLL4.

In one embodiment, an immunogenic composition of the invention is a population of DLL4-DC activated T cells. Methods of generating a population of DLL4-DC activated T cells have been described elsewhere herein.

In one embodiment, an immunogenic composition of the invention is a T cell engineered to express DLL4. In one embodiment, a DLL4 expressing T cell of the invention further comprises one or more cell receptor that has been genetically engineered. In one embodiment, a receptor is a T cell receptor (TCR). In one embodiment, a receptor is a chimeric antigen receptor (CAR), and the T cell of the invention is a CAR T cell.

In one embodiment, the receptor of the invention binds to a specific antigenic peptide. In one embodiment, the tumor antigen comprises one or more antigenic cancer epitopes associated with a malignant tumor. Tumor antigens appropriate for targeting with an engineered CAR/TCR include those discussed elsewhere herein.

### Sources of T Cells

Prior to expansion, a source of T cells is obtained from a subject. The term “subject” is intended to include living organisms in which an immune response can be elicited (e.g., mammals). Examples of subjects include humans, dogs, cats, mice, rats, and transgenic species thereof. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present invention, any number of T cell lines available in the art, may be used. In certain embodiments of the present invention, T cells can be obtained from a unit of blood collected from a subject using any number of techniques known to the skilled artisan, such as ficoll separation. In one embodiment, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In one embodiment of the invention, the cells are washed with phosphate buffered saline (PBS). In an alternative embodiment, the wash solution lacks calcium and may lack magnesium or may lack many if not all divalent cations. Again, surprisingly, initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca-free, Mg-free PBS, PlasmaLyte A, or other saline solution with or without buffer. Alternatively, the undesirable components of the apheresis sample may be removed and the cells directly resuspended in culture media.

In another embodiment, T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD28⁺, CD4⁺, CD8⁺, CD45RA⁺, and CD45RO⁺ T cells, can be further isolated by positive or negative selection techniques. For example, in one embodiment, T cells are isolated by incubation with anti-CD³/anti-CD28 (i.e., 3×28)-conjugated beads, such as DYNABEADS® M-450 CD3/CD28 T, for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another embodiment, the time period is 10 to 24 hours. In one embodiment, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used in the context of this invention. In certain embodiments, it may be desirable to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4⁺ cells by negative selection, a monoclonal antibody cocktail typically includes antibodies to CD14, CD20, CD11b, CD16, HLA-DR, and CD8. In certain embodiments, it may be desirable to enrich for or positively select for regulatory T cells which typically express CD4⁺, CD25⁺, CD62^{hi}, GITR⁺, and FoxP3⁺. Alternatively, in certain embodiments, T regulatory cells are depleted by anti-C25 conjugated beads or other similar method of selection.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc.). Such populations of cells may have therapeutic value and would be desirable to obtain. For example, using high concentration of cells allows more efficient selection of CD8⁺ T cells that normally have weaker CD28 expression.

In a related embodiment, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4⁺ T cells express higher levels of CD28 and are more efficiently captured than CD8⁺ T cells in dilute concentrations. In one embodiment, the concentration of cells used is 5×10⁶/ml. In other embodiments, the concentration used can be from about 1×10⁵/ml to 1×10⁶/ml, and any integer value in between.

In other embodiments, the cells may be incubated on a rotator for varying lengths of time at varying speeds at either 2-10° C. or at room temperature.

If desired or necessary, monocyte populations (i.e., CD14⁺ cells) may be depleted from blood preparations prior to ex vivo expansion by a variety of methodologies, including anti-CD14 coated beads or columns, or utilization of the phagocytotic activity of these cells to facilitate removal, or by the use of counterflow centrifugal elutriation. Accordingly, in one embodiment, the invention uses paramagnetic particles of a size sufficient to be engulfed by phagocytotic monocytes. In certain embodiments, the paramagnetic particles are commercially available beads, for example, those produced by Dynal AS under the trade name Dynabeads™. Exemplary Dynabeads™ in this regard are M-280, M-450, and M-500. In one aspect, other non-specific cells are removed by coating the paramagnetic particles with “irrelevant” proteins (e.g., serum proteins or antibodies). Irrelevant proteins and antibodies include those proteins and antibodies or fragments thereof that do not specifically target the T cells to be expanded. In certain embodiments the irrelevant beads include beads coated with sheep anti-mouse antibodies, goat anti-mouse antibodies, and human serum albumin.

In brief, such depletion of monocytes is performed by preincubating PBMC isolated from whole blood or apheresed peripheral blood with one or more varieties of irrelevant or non-antibody coupled paramagnetic particles at any amount that allows for removal of monocytes (approximately a 20:1 bead:cell ratio) for about 30 minutes to 2 hours at 22 to 37 degrees C., followed by magnetic removal of cells which have attached to or engulfed the paramagnetic particles. Such separation can be performed using standard methods available in the art. For example, any magnetic separation methodology may be used including a variety of which are commercially available, (e.g., DYNAL® Magnetic Particle Concentrator (DYNAL MPC®)). Assurance of requisite depletion can be monitored by a variety of methodologies known to those of ordinary skill in the art, including flow cytometric analysis of CD14 positive cells, before and after said depletion.

T cells for stimulation can also be frozen after the washing step, which does not require the monocyte-removal step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In certain embodiments, cryopreserved cells are thawed and washed as described herein and allowed to rest for one hour at room temperature prior to activation using the methods of the present invention.

Also contemplated in the context of the invention is the collection of blood samples or leukapheresis product from a subject at a time period prior to when the expanded cells as described herein might be needed. As such, the source of the cells to be expanded can be collected at any time point necessary, and desired cells, such as T cells, isolated and frozen for later use in T cell therapy for any number of diseases or conditions that would benefit from T cell therapy, such as those described herein. In one embodiment a blood sample or a leukapheresis is taken from a generally healthy subject. In certain embodiments, a blood sample or a leukapheresis is taken from a generally healthy subject who is at risk of developing a disease, but who has not yet developed a disease, and the cells of interest are isolated and frozen for later use. In certain embodiments, the T cells may be expanded, frozen, and used at a later time. In certain embodiments, samples are collected from a patient shortly after diagnosis of a particular disease as described herein but prior to any treatments. In a further embodiment, the cells are isolated from a blood sample or a leukapheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxin, fludaribine, cyclosporin, FK506, rapamycin, mycophenolic acid, steroids, FR901228, and irradiation. These drugs inhibit either the calcium dependent phosphatase calcineurin (cyclosporine and FK506) or inhibit the p70S6 kinase that is important for growth factor induced signaling (rapamycin). (Liu et al., Cell 66:807-815, 1991; Henderson et al., Immun. 73:316-321, 1991; Bierer et al., Curr. Opin. Immun. 5:763-773, 1993; Isoniemi (supra)). In a further embodiment, the cells are isolated for a patient and frozen for later use in conjunction with (e.g., before, simultaneously or following) bone marrow or stem cell transplantation, T cell ablative therapy using either chemotherapy agents such as, fludarabine, external-beam radiation therapy (XRT), cyclophosphamide, or antibodies such as OKT3 or CAMPATH. In another embodiment, the cells are isolated prior to and can be frozen for later use for treatment following B-cell ablative therapy such as agents that react with CD20, e.g., Rituxan.

In a further embodiment of the present invention, T cells are obtained from a patient directly following treatment. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a state for enhanced engraftment and in vivo expansion. Thus, it is contemplated within the context of the present invention to collect blood cells, including T cells, dendritic cells, or other cells of the hematopoetic lineage, during this recovery phase. Further, in certain embodiments, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

### Activation and Expansion of T Cells

Whether prior to or after genetic modification of the T cells to express DLL4 and/or a desirable TCR/CAR, the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

Generally, the T cells of the invention are expanded by contact with a surface having attached thereto an agent that stimulates a CD3/TCR complex associated signal and a ligand that stimulates a co-stimulatory molecule on the surface of the T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody. Examples of an anti-CD28 antibody include 9.3, B-T3, XR-CD28 (Diaclone, Besançon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol Meth. 227(1-2):53-63, 1999).

In certain embodiments, the primary stimulatory signal and the co-stimulatory signal for the T cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In one embodiment, the agent providing the co-stimulatory signal is bound to a cell surface and the agent providing the primary activation signal is in solution or coupled to a surface. In certain embodiments, both agents can be in solution. In another embodiment, the agents may be in soluble form, and then cross-linked to a surface, such as a cell expressing Fc receptors or an antibody or other binding agent which will bind to the agents. In this regard, see for example, U.S. Patent Application Publication Nos. 20040101519 and 20060034810 for artificial antigen presenting cells (aAPCs) that are contemplated for use in activating and expanding T cells in the present invention.

In one embodiment, the two agents are immobilized on beads, either on the same bead, i.e., “cis,” or to separate beads, i.e., “trans.” By way of example, the agent providing the primary activation signal is an anti-CD3 antibody or an antigen-binding fragment thereof and the agent providing the co-stimulatory signal is an anti-CD28 antibody or antigen-binding fragment thereof; and both agents are co-immobilized to the same bead in equivalent molecular amounts. In one embodiment, a 1:1 ratio of each antibody bound to the beads for CD4+ T cell expansion and T cell growth is used. In certain aspects of the present invention, a ratio of anti CD3:CD28 antibodies bound to the beads is used such that an increase in T cell expansion is observed as compared to the expansion observed using a ratio of 1:1. In one particular embodiment an increase of from about 1 to about 3 fold is observed as compared to the expansion observed using a ratio of 1:1. In one embodiment, the ratio of CD3:CD28 antibody bound to the beads ranges from 100:1 to 1:100 and all integer values there between. In one aspect of the present invention, more anti-CD28 antibody is bound to the particles than anti-CD3 antibody, i.e., the ratio of CD3:CD28 is less than one. In certain embodiments of the invention, the ratio of anti CD28 antibody to anti CD3 antibody bound to the beads is greater than 2:1. In one particular embodiment, a 1:100 CD3:CD28 ratio of antibody bound to beads is used. In another embodiment, a 1:75 CD3:CD28 ratio of antibody bound to beads is used. In a further embodiment, a 1:50 CD3:CD28 ratio of antibody bound to beads is used. In another embodiment, a 1:30 CD3:CD28 ratio of antibody bound to beads is used. In one embodiment, a 1:10 CD3:CD28 ratio of antibody bound to beads is used. In another embodiment, a 1:3 CD3:CD28 ratio of antibody bound to the beads is used. In yet another embodiment, a 3:1 CD3:CD28 ratio of antibody bound to the beads is used.

Ratios of particles to cells from 1:500 to 500:1 and any integer values in between may be used to stimulate T cells or other target cells. As those of ordinary skill in the art can readily appreciate, the ratio of particles to cells may depend on particle size relative to the target cell. For example, small sized beads could only bind a few cells, while larger beads could bind many. In certain embodiments the ratio of cells to particles ranges from 1:100 to 100:1 and any integer values in-between and in further embodiments the ratio comprises 1:9 to 9:1 and any integer values in between, can also be used to stimulate T cells. The ratio of anti-CD3- and anti-CD28-coupled particles to T cells that result in T cell stimulation can vary as noted above, however certain exemplary values include 1:100, 1:50, 1:40, 1:30, 1:20, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, and 15:1 with one exemplary ratio being at least 1:1 particles per T cell. In one embodiment, a ratio of particles to cells of 1:1 or less is used. In one particular embodiment, a particle:cell ratio is 1:5. In further embodiments, the ratio of particles to cells can be varied depending on the day of stimulation. For example, in one embodiment, the ratio of particles to cells is from 1:1 to 10:1 on the first day and additional particles are added to the cells every day or every other day thereafter for up to 10 days, at final ratios of from 1:1 to 1:10 (based on cell counts on the day of addition). In one particular embodiment, the ratio of particles to cells is 1:1 on the first day of stimulation and adjusted to 1:5 on the third and fifth days of stimulation. In another embodiment, particles are added on a daily or every other day basis to a final ratio of 1:1 on the first day, and 1:5 on the third and fifth days of stimulation. In another embodiment, the ratio of particles to cells is 2:1 on the first day of stimulation and adjusted to 1:10 on the third and fifth days of stimulation. In another embodiment, particles are added on a daily or every other day basis to a final ratio of 1:1 on the first day, and 1:10 on the third and fifth days of stimulation. One of skill in the art will appreciate that a variety of other ratios may be suitable for use in the present invention. In particular, ratios will vary depending on particle size and on cell size and type.

In further embodiments of the present invention, the cells, such as T cells, are combined with agent-coated beads, the beads and the cells are subsequently separated, and then the cells are cultured. In an alternative embodiment, prior to culture, the agent-coated beads and cells are not separated but are cultured together. In a further embodiment, the beads and cells are first concentrated by application of a force, such as a magnetic force, resulting in increased ligation of cell surface markers, thereby inducing cell stimulation.

By way of example, cell surface proteins may be ligated by allowing paramagnetic beads to which anti-CD3 and anti-CD28 are attached (3×28 beads) to contact the T cells. In one embodiment the cells (for example, 10⁴ to 10⁹ T cells) and beads (for example, DYNABEADS® M-450 CD3/CD28 T paramagnetic beads at a ratio of 1:1) are combined in a buffer, for example in PBS without divalent cations such as, calcium and magnesium. Again, those of ordinary skill in the art can readily appreciate any cell concentration may be used. For example, the target cell may be very rare in the sample and comprise only 0.01% of the sample or the entire sample (i.e., 100%) may comprise the target cell of interest. Accordingly, any cell number is within the context of the present invention. In certain embodiments, it may be desirable to significantly decrease the volume in which particles and cells are mixed together (i.e., increase the concentration of cells), to ensure maximum contact of cells and particles. For example, in one embodiment, a concentration of about 2 billion cells/ml is used. In another embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells. Such populations of cells may have therapeutic value and would be desirable to obtain in certain embodiments. For example, using high concentration of cells allows more efficient selection of CD8+ T cells that normally have weaker CD28 expression.

In one embodiment of the present invention, the mixture may be cultured for several hours (about 3 hours) to about 14 days or any hourly integer value in between. In another embodiment, the mixture may be cultured for 21 days. In one embodiment of the invention the beads and the T cells are cultured together for about eight days. In another embodiment, the beads and T cells are cultured together for 2-3 days. Several cycles of stimulation may also be desired such that culture time of T cells can be 60 days or more. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-γ, IL-4, IL-7, GM-CSF, IL-10, IL-12, IL-15, TGFβ, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO2).

In one embodiment, the invention provides DLL4 protein-conjugated magnetic beads, which can be used for regulating T cell expansion and function. For example, DLL4 protein-conjugated magnetic beads can be used together with anti-CD3-Ab and anti-CD28 Ab to expand T cells in cultures for the purpose of adoptive T-cell therapy for cancer, hematopoietic stem cell transplantation and autoimmune diseases.

In one embodiment, the DLL4-protein-conjugated protein complex can be used as an immune adjuvant.

T cells that have been exposed to varied stimulation times may exhibit different characteristics. For example, typical blood or apheresed peripheral blood mononuclear cell products have a helper T cell population (TH, CD4+) that is greater than the cytotoxic or suppressor T cell population (TC, CD8+). Ex vivo expansion of T cells by stimulating CD3 and CD28 receptors produces a population of T cells that prior to about days 8-9 consists predominately of TH cells, while after about days 8-9, the population of T cells comprises an increasingly greater population of TC cells. Accordingly, depending on the purpose of treatment, infusing a subject with a T cell population comprising predominately of TH cells may be advantageous. Similarly, if an antigen-specific subset of TC cells has been isolated it may be beneficial to expand this subset to a greater degree.

Further, in addition to CD4 and CD8 markers, other phenotypic markers vary significantly, but in large part, reproducibly during the course of the cell expansion process. Thus, such reproducibility enables the ability to tailor an activated T cell product for specific purposes.

### Genetic Modification

The present invention encompasses a DNA construct comprising sequences of DLL4. The nucleic acid sequences coding for the desired molecules can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

The present invention also provides vectors in which a DNA of the present invention is inserted. Vectors derived from retroviruses, such as the lentivirus, are suitable tools to achieve long-term gene transfer since they allow long-term, stable integration of a transgene or transgenes and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

In one embodiment, expression of natural or synthetic nucleic acids encoding DLL4 may be achieved by operably linking a nucleic acid encoding the polypeptide or portions thereof to one or more promoters, and incorporating the construct into an expression vector. The vectors can be suitable for replication and integration eukaryotes. Typical cloning vectors contain transcription and translation terminators, initiation sequences, and promoters useful for regulation of the expression of the desired nucleic acid sequence.

The expression constructs of the present invention may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In another embodiment, the invention provides a gene therapy vector.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription. One example of an element known to increase expression levels is the woodchuck hepatitis post-transcriptional regulatory element (WPRE) (see, for example, Klein et al., 2006, Gene 372:153-161).

In some embodiments, the nucleic acid construct of the invention are multicystronic constructs that permit the expression of multiple transgenes (e.g., DLL4, TCR-α, TCR-β, CAR, etc.) under the control of a single promoter. In some embodiments, the transgenes (e.g., DLL4, TCR-α, TCR-β, CAR, etc.) are separated by a self-cleaving 2A peptide. Examples of 2A peptides useful in the nucleic acid constructs of the invention include F2A, P2A, T2A and E2A (see for example, Kim et al., PLoS One 6:e18556; Carey et al., 2009, PNAS 106:157-162; Szymczak et al., 2004, Nature Biotechnology 22:589-594).

In other embodiments of the invention, the nucleic acid construct of the invention is a multicystronic construct comprising two promoters; one promoter driving the expression of DLL4, and the other promoter driving the expression of the TCR/CAR. In some embodiments, the dual promoter constructs of the invention are uni-directional. In other embodiments, the dual promoter constructs of the invention are bi-directional.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In order to assess the expression of DLL4 or a TCR/CAR polypeptide or portions thereof, the expression vector to be introduced into a cell can also contain either a selectable marker gene or a reporter gene or both to facilitate identification and selection of expressing cells from the population of cells sought to be transfected or transduced through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEB S Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York). An exemplary method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

### RNA-Engineered Cells

An advantage of the methods of the invention is that RNA transfection is essentially transient and a vector-free: An RNA transgene can be delivered to an immune and expressed therein following a brief in vitro cell activation, as a minimal expressing cassette without the need for any additional viral sequences. Under these conditions, integration of the transgene into the host cell genome is unlikely. Cloning of cells is not necessary because of the efficiency of transfection of the RNA and its ability to uniformly modify the entire lymphocyte population. Thus, cells containing an RNA construct introduced according to the disclosed method can be used therapeutically.

Immune therapy with in vitro-transcribed RNA (IVT-RNA) makes use of two different strategies both of which have been successively tested in various animal models. Cells are transfected with in vitro-transcribed RNA by means of lipofection or electroporation and administered to the subject. In one embodiment, it is desirable to stabilize IVT-RNA using various modifications in order to achieve prolonged expression of transferred IVT-RNA.

Some IVT vectors are known in the literature which are utilized in a standardized manner as template for in vitro transcription and which have been genetically modified in such a way that stabilized RNA transcripts are produced. Currently protocols used in the art are based on a plasmid vector with the following structure: a 5′ RNA polymerase promoter enabling RNA transcription, followed by a gene of interest which is flanked either 3′ and/or 5′ by untranslated regions (UTR), and a 3′ polyadenyl cassette containing a chain of A nucleotides. Prior to in vitro transcription, the circular plasmid is linearized downstream of the polyadenyl cassette by type II restriction enzymes (recognition sequence corresponds to cleavage site). The polyadenyl cassette thus corresponds to the later poly(A) sequence in the transcript. As a result of this procedure, some nucleotides remain as part of the enzyme cleavage site after linearization and extend or mask the poly(A) sequence at the 3′ end. It is not clear, whether this nonphysiological overhang affects the amount of protein produced intracellularly from such a construct.

RNA has several advantages over more traditional plasmid or viral approaches. Gene expression from an RNA source does not require transcription and the protein product is produced rapidly after the transfection. Further, since the RNA has to only gain access to the cytoplasm, rather than the nucleus, and therefore typical transfection methods result in an extremely high rate of transfection. In addition, plasmid based approaches require that the promoter driving the expression of the gene of interest be active in the cells under study.

In another aspect, the RNA construct can be delivered into the cells by electroporation. See, e.g., the formulations and methodology of electroporation of nucleic acid constructs into mammalian cells as taught in US 2004/0014645, US 2005/0052630A1, US 2005/0070841A1, US 2004/0059285A1, US 2004/0092907A1. The various parameters including electric field strength required for electroporation of any known cell type are generally known in the relevant research literature as well as numerous patents and applications in the field. See e.g., U.S. Pat. Nos. 6,678,556, 7,171,264, and 7,173,116. Apparatus for therapeutic application of electroporation are available commercially, e.g., the MedPulser™ DNA Electroporation Therapy System (Inovio/Genetronics, San Diego, Calif.), and are described in patents such as U.S. Pat. Nos. 6,567,694; 6,516,223, 5,993,434, 6,181,964, 6,241,701, and 6,233,482; electroporation may also be used for transfection of cells in vitro as described e.g. in US20070128708A1. Electroporation may also be utilized to deliver nucleic acids into cells in vitro. Accordingly, electroporation-mediated administration into cells of nucleic acids including expression constructs utilizing any of the many available devices and electroporation systems known to those of skill in the art presents an exciting new means for delivering an RNA of interest to a target cell.

### Therapeutic Application

The present invention encompasses DLL4-expressing immune cells and immune cells that have been stimulated using DLL4-expressing immune cells. In one embodiment, the present invention encompasses an immune cell that expresses DLL4. In one embodiment, the present invention encompasses an immune cell that is stimulated by an immune cell that expresses DLL4. In one embodiment, the DLL4-expressing cell is a dendritic cell. In one embodiment, the DLL4-expressing cell is a T cell. Together the cells of the invention are referred to generally as “DLL4-ICs”. The present invention also provides a method for stimulating a T cell-mediated immune response to a target cell population or tissue in a mammal comprising the step of administering to the mammal a DLL4-IC.

In one embodiment, the present invention encompasses an immune cell transduced with a lentiviral vector (LV). For example, the LV encodes DLL4. In one embodiment, the immune cell is a dendritic cell transduced with a LV that encodes DLL4. In one embodiment, the immune cell is further transduced with a TCR or CAR with binding specificity to a particular antigen. Therefore, in some instances, the immune cell can elicit a TCR-mediated T-cell response or a CAR-mediated T-cell response.

The invention provides the use of a TCR/CAR to redirect the specificity of a DLL4-IC to at least one tumor antigens. Thus, the present invention also provides a method for stimulating a T cell-mediated immune response to a target cell population or tissue in a mammal comprising the step of administering to the mammal a DLL4-IC that expresses a TCR/CAR, wherein the TCR has binding specificity to a predetermined target antigen, or a CAR, wherein the CAR comprises a binding moiety that specifically interacts with a predetermined target.

In one embodiment, the present invention includes a type of cellular therapy where T cells are genetically modified to express both DLL4 and a TCR/CAR and the DLL4-IC is infused to a recipient in need thereof. The infused cell is able to kill tumor cells in the recipient.

In one embodiment, the DLL4-IC of the invention can undergo robust in vivo T cell expansion and can persist for an extended amount of time. In another embodiment, the DLL4-IC of the invention evolve into specific memory T cells that can be reactivated to inhibit any additional tumor formation or growth. Without wishing to be bound by any particular theory, DLL4-IC may differentiate in vivo into a central memory-like state upon encounter and subsequent elimination of target cells expressing the surrogate antigen.

Without wishing to be bound by any particular theory, the anti-tumor immunity response elicited by the DLL4-IC may be an active or a passive immune response. In addition, the DLL4-IC mediated immune response may be part of an adoptive immunotherapy approach in which DLL4-IC induce an immune response specific an antigen.

Cancers that may be treated include tumors that are not vascularized, or not yet substantially vascularized, as well as vascularized tumors. The cancers may comprise non-solid tumors (such as hematological tumors, for example, leukemias and lymphomas) or may comprise solid tumors. Types of cancers to be treated with the DLL4-IC of the invention include, but are not limited to, carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included.

However, the invention should not be construed to be limited to solely to the antigen targets and diseases disclosed herein. Rather, the invention should be construed to include any antigenic target that is associated with a disease where a DLL4-IC can be used to treat the disease.

In one embodiment, a cell may be isolated from a culture, tissue, organ or organism and administered to a mammal as a cellular vaccine. Thus, the present invention contemplates a “cellular vaccine.” Of course, the cell may also express one or more additional vaccine components, such as immunomodulators or adjuvants. The vaccine may comprise all or part of the cell. In one embodiment, the cellular vaccine of the present invention comprises a human APC and in an exemplary embodiment, the APC is a DC. In another embodiment, the cellular vaccine of the present invention comprises a human T cell.

The cellular vaccine can comprise of an APC or T cell that has been genetically engineered according to the present invention to express DLL4. The DLL4-expressing cell can further be transfected with a nucleic acid encoding an antigen to generate an antigen-loaded cell. In one embodiment, a DLL4-expressing DC can be pulsed with an immunostimulatory protein comprising an antigen to generate an antigen-loaded, DLL4-expressing DC. Based on the present disclosure, the DLL4-expressing DC can be pulsed by any method using any type of antigen to load the antigen. In addition, a DLL4-expressing DC can be pulsed by any method prior to, concurrently with or following infection, transfection or transduction of the DC with a DLL4-encoding nucleic acid sequence of the present invention.

As disclosed elsewhere herein, a cell can be pulsed with an antigen using various methods. An antigen of the present invention contains at least one epitope, wherein said epitope is capable of eliciting an immune response in a mammal. In one embodiment, the antigen is expressed by an expression vector. In another embodiment, the antigen is an isolated polypeptide. In one embodiment, the antigen is associated with a disease selected from the group consisting of an infectious disease, a cancer and an autoimmune disease. A number of non-limiting exemplary antigens useful for pulsing the cells of the invention are disclosed elsewhere herein. The antigen can be in the form of at least one or more of the following: a tumor lysate, a protein, a peptide, an mRNA, a DNA, expressed from a vector, a liposome and the like.

The DLL4-IC of the invention may also serve as a type of vaccine for ex vivo immunization and/or in vivo therapy in a mammal. In one embodiment, the mammal is a human. In one embodiment, with respect to ex vivo immunization, at least one of the following occurs in vitro prior to administering the cell into a mammal: i) expansion of the cells, ii) introducing a nucleic acid encoding DLL4 to the cells, and iii) pulsing of the cells with at least one antigen or introduction of a nucleic acid encoding at least one antigen to the cells.

Ex vivo procedures are well known in the art. Briefly, in one embodiment, cells are isolated from a mammal (e.g., a human) and genetically modified (i.e., transduced or transfected in vitro) with a vector comprising a nucleic acid sequence encoding DLL4. The DLL4-IC can be administered to a mammalian recipient to provide a therapeutic benefit. The mammalian recipient may be a human and the DLL4-IC cell can be autologous with respect to the recipient. Alternatively, the cells can be allogeneic, syngeneic or xenogeneic with respect to the recipient.

In addition to using a cell-based vaccine in terms of ex vivo immunization, the present invention also provides compositions and methods for in vivo immunization to elicit an immune response directed against an antigen in a patient.

Generally, the cells activated and expanded as described herein may be utilized in the treatment and prevention of diseases that arise in individuals who are immunocompromised. In particular, the DLL4-IC of the invention are used in the treatment cancer. In certain embodiments, the cells of the invention are used in the treatment of patients at risk for developing cancer. Thus, the present invention provides methods for the treatment or prevention of cancer comprising administering to a subject in need thereof, a therapeutically effective amount of the DLL4-IC of the invention.

The DLL4-IC of the present invention may be administered either alone, or as a pharmaceutical composition in combination with diluents and/or with other components such as IL-2 or other cytokines or cell populations. Briefly, pharmaceutical compositions of the present invention may comprise a target cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. In one embodiment, compositions of the present invention are formulated for intravenous administration.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

When “an immunologically effective amount”, “an anti-tumor effective amount”, “an tumor-inhibiting effective amount”, or “therapeutic amount” is indicated, the precise amount of the compositions of the present invention to be administered can be determined by a physician with consideration of individual differences in age, weight, tumor size, extent of infection or metastasis, and condition of the patient (subject). In one embodiment, a pharmaceutical composition comprising the DLL4-IC described herein may be administered at a dosage of 10⁴ to 10⁹ cells/kg body weight, for example at a dosage of 10⁵ to 10⁶ cells/kg body weight, including all integer values within those ranges. DLL4-IC compositions may also be administered multiple times at these dosages. The DLL4-IC can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

In certain embodiments, it may be desired to administer activated T cells to a subject and then subsequently redraw blood (or have an apheresis performed), activate T cells therefrom according to the present invention, and reinfuse the patient with these activated and expanded T cells. This process can be carried out multiple times every few weeks. In certain embodiments, T cells can be activated from blood draws of from 10 cc to 400 cc. In certain embodiments, T cells are activated from blood draws of 20 cc, 30 cc, 40 cc, 50 cc, 60 cc, 70 cc, 80 cc, 90 cc, or 100 cc. Not to be bound by theory, using this multiple blood draw/multiple reinfusion protocol may serve to select out certain populations of T cells.

The administration of the DLL4-IC compositions may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous (i.v.) injection, or intraperitoneally. In one embodiment, the DLL4-IC compositions of the present invention are administered to a patient by intradermal or subcutaneous injection. In another embodiment, the DLL4-IC compositions of the present invention are administered by i.v. injection. The compositions of T cells may be injected directly into a tumor, lymph node, or site of infection.

In certain embodiments of the present invention, cells activated and expanded using the methods described herein, or other methods known in the art where immune cells are expanded to therapeutic levels, are administered to a patient in conjunction with (e.g., before, simultaneously or following) any number of relevant treatment modalities, including but not limited to treatment with agents such as antiviral therapy, cidofovir and interleukin-2, Cytarabine (also known as ARA-C) or natalizumab treatment for MS patients or efalizumab treatment for psoriasis patients or other treatments for PML patients. In further embodiments, the DLL4-IC of the invention may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. These drugs inhibit either the calcium dependent phosphatase calcineurin (cyclosporine and FK506) or inhibit the p70S6 kinase that is important for growth factor induced signaling (rapamycin) (Liu et al., Cell 66:807-815, 1991; Henderson et al., Immun. 73:316-321, 1991; Bierer et al., Curr. Opin. Immun. 5:763-773, 1993). In a further embodiment, the DLL4-IC of the present invention are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In another embodiment, the DLL4-IC of the present invention are administered following B-cell ablative therapy such as agents that react with CD20, e.g., Rituxan. For example, in one embodiment, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

The dosage of the above treatments to be administered to a patient will vary with the precise nature of the condition being treated and the recipient of the treatment. The scaling of dosages for human administration can be performed according to art-accepted practices. The dose for CAMPATH, for example, will generally be in the range 1 to about 100 mg for an adult patient, usually administered daily for a period between 1 and 30 days. In one embodiment, a daily dose is 1 to 10 mg per day although in some instances larger doses of up to 40 mg per day may be used (described in U.S. Pat. No. 6,120,766).

### Methods

In one embodiment, the present invention includes a method of enhancing the immune response in a mammal comprising the steps of: generating immature DCs from monocytes obtained from a mammal (e.g., a patient); pulsing the immature DCs with a composition comprising an antigenic composition; genetically modifying the DCs to express DLL4, activating the antigen loaded DLL4-DCs and then administering the activated, antigen loaded DLL4-DCs to a mammal in need thereof.

In one embodiment, the present invention includes a method of enhancing the immune response in a mammal comprising the steps of: generating immature DCs from bone marrow cells obtained from a mammal (e.g., a patient); pulsing the immature DCs with a composition comprising an antigenic composition; activating the antigen loaded DCs with Flt3L and at least one TLR agonist and then administering the activated, antigen loaded DLL4-DCs to a mammal in need thereof.

In one embodiment, the present invention includes a method of enhancing the immune response in a mammal comprising the steps of: generating immature DCs from bone marrow cells obtained from a mammal (e.g., a patient); pulsing the immature DCs with a composition comprising an antigenic composition; activating the antigen loaded DCs with Flt3L and at least one TLR agonist, contacting a population of naïve T cells with the activated, antigen loaded DLL4-DCs, and then administering the DLL4-DC stimulated T cells to a mammal in need thereof.

In one embodiment, the present invention includes a method of enhancing the immune response in a mammal comprising the steps of: isolating naïve T cells from a mammal (e.g., a patient); genetically modifying the T cells to express DLL4 and optionally an antigen binding CAR/TCR; activating the DLL4-T cells, and then administering the DLL4-T cells to a mammal in need thereof.

The DLL4-DCs can be administered to a mammalian recipient to provide a therapeutic benefit. The mammalian recipient may be a human and the cells can be autologous with respect to the recipient. Alternatively, the cells can be allogeneic, syngeneic or xenogeneic with respect to the recipient.

### Gene Therapy Administration

One skilled in the art recognizes that different methods of delivery may be utilized to administer a vector into a cell. Examples include: (1) methods utilizing physical means, such as electroporation (electricity), a gene gun (physical force) or applying large volumes of a liquid (pressure); and (2) methods wherein said vector is complexed to another entity, such as a liposome, aggregated protein or transporter molecule.

Furthermore, the actual dose and schedule can vary depending on whether the compositions are administered in combination with other pharmaceutical compositions, or depending on interindividual differences in pharmacokinetics, drug disposition, and metabolism. Similarly, amounts can vary in in vitro applications depending on the particular cell line utilized (e.g., based on the number of vector receptors present on the cell surface, or the ability of the particular vector employed for gene transfer to replicate in that cell line). Furthermore, the amount of vector to be added per cell will likely vary with the length and stability of the therapeutic gene inserted in the vector, as well as also the nature of the sequence, and is particularly a parameter which needs to be determined empirically, and can be altered due to factors not inherent to the methods of the present invention (for instance, the cost associated with synthesis). One skilled in the art can easily make any necessary adjustments in accordance with the exigencies of the particular situation.

Cells containing the therapeutic agent may also contain a suicide gene i.e., a gene which encodes a product that can be used to destroy the cell. In many gene therapy situations, it is desirable to be able to express a gene for therapeutic purposes in a host, cell but also to have the capacity to destroy the host cell at will. The therapeutic agent can be linked to a suicide gene, whose expression is not activated in the absence of an activator compound. When death of the cell in which both the agent and the suicide gene have been introduced is desired, the activator compound is administered to the cell thereby activating expression of the suicide gene and killing the cell. Examples of suicide gene/prodrug combinations which may be used are herpes simplex virus-thymidine kinase (HSV-tk) and ganciclovir, acyclovir; oxidoreductase and cycloheximide; cytosine deaminase and 5-fluorocytosine; thymidine kinase thymidilate kinase (Tdk::Tmk) and AZT; and deoxycytidine kinase and cytosine arabinoside.

### Vaccine Formulations

The present invention further includes vaccine formulations suitable for use in immunotherapy. In certain embodiments, vaccine formulations are used for the prevention and/or treatment of a disease, such as cancer and infectious diseases. In one embodiment, the administration to a patient of a vaccine in accordance with the present invention for the prevention and/or treatment of cancer can take place before or after a surgical procedure to remove the cancer, before or after a chemotherapeutic procedure for the treatment of cancer, and before or after radiation therapy for the treatment of cancer and any combination thereof. In other embodiments, the vaccine formulations may be administrated to a patient in conjunction or combination with another composition or pharmaceutical product. It should be appreciated that the present invention can also be used to prevent cancer in individuals without cancer, but who might be at risk of developing cancer.

The administration of a cancer vaccine prepared in accordance with the present invention, is broadly applicable to the prevention or treatment of cancer, determined in part by the selection of antigens forming part of the cancer vaccine. Cancers that can be suitably treated in accordance with the practices of the present invention include, without limitation, cancers of the lung, breast, ovary, cervix, colon, head and neck, pancreas, prostate, stomach, bladder, kidney, bone, liver, esophagus, brain, testicle, uterus and the various leukemias and lymphomas.

In one embodiment, vaccines in accordance with this invention can be derived from the tumor or cancer cells to be treated. For example, in the treatment of lung cancer, the lung cancer cells would be treated as described hereinabove to produce a lung cancer vaccine. Similarly, breast cancer vaccine, colon cancer vaccine, pancreas cancer vaccine, stomach cancer vaccine, bladder cancer vaccine, kidney cancer vaccine and the like, would be produced and employed as immunotherapeutic agents in accordance with the practices for the prevention and/or treatment of the tumor or cancer cell from which the vaccine was produced.

In another embodiment, vaccines in accordance with the present invention could, as stated, also be prepared to treat various infectious diseases which affect mammals, by collecting the relevant antigens shed into a culture medium by the pathogen. As there is heterogenecity in the type of immunogenic and protective antigens expressed by different varieties of organisms causing the same disease, polyvalent vaccines can be prepared by preparing the vaccine from a pool of organisms expressing the different antigens of importance.

In another embodiment of the present invention, the vaccine can be administered by intranodal injection into groin nodes. Alternatively, and depending on the vaccine target, the vaccine can be intradermally or subcutaneously administered to the extremities, arms and legs, of the patients being treated. Although this approach is generally satisfactory for melanoma and other cancers, including the prevention or treatment of infectious diseases, other routes of administration, such as intramuscularly or into the blood stream may also be used.

Additionally, the vaccine can be given together with adjuvants and/or immuno-modulators to boost the activity of the vaccine and the patient's response. Such adjuvants and/or immuno-modulators are understood by those skilled in the art, and are readily described in available published literature.

As contemplated herein, and depending on the type of vaccine being generated, the production of vaccine can, if desired, be scaled up by culturing cells in bioreactors or fermentors or other such vessels or devices suitable for the growing of cells in bulk. In such apparatus, the culture medium would be collected regularly, frequently or continuously to recover therefrom any materials or antigens before such materials or antigens are degraded in the culture medium.

If desired, devices or compositions containing the vaccine or antigens produced and recovered, in accordance with the present invention, and suitable for sustained or intermittent release could be, in effect, implanted in the body or topically applied thereto for a relatively slow or timed release of such materials into the body.

Other steps in vaccine preparation can be individualized to satisfy the requirements of particular vaccines. Such additional steps will be understood by those skilled in the art. For example, certain collected antigenic materials may be concentrated and in some cases treated with detergent and ultracentrifuged to remove transplantation alloantigens.

These methods described herein are by no means all-inclusive, and further methods to suit the specific application will be apparent to the ordinary skilled artisan. Moreover, the effective amount of the compositions can be further approximated through analogy to compounds known to exert the desired effect.

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, describe certain exemplary embodiments of the present invention, and are not to be construed as limiting in any way the remainder of the disclosure.

### Example 1

### Programming of Donor T Cells Using Allogeneic Delta-Like Ligand 4-Positive Dendritic Cells to Reduce Graft-Versus-Host Disease in Mice

Allogeneic hematopoietic stem cell transplantation (HSCT) is an effective cellular therapy for hematological malignancies. A primary barrier that limits its success is acute graft-versus host disease (GVHD). (Choi et al., Nat Rev Clin Oncol, 2014, 11:536-46; Choi et al., Blood, 2010, 116:129-139; Bleakley and Riddell, Immunol Cell Biol, 2011, 89:396-407; Blazar et al., Nat Rev Immunol, 2012, 12:443-458; Shlomchik, Nat Rev Immunol, 2007, 7:340-352). GVHD is caused by donor T cells that recognize and react to histocompatibility differences between host and donor cells. GVHD is initiated by priming of donor T cells by host antigen-presenting cells and followed by robust proliferation and differentiation of alloreactive T cells that mediate tissue injury (Blazar et al., Nat Rev Immunol, 2012, 12:443-458; Shlomchik, Nat Rev Immunol, 2007, 7:340-352). Thus, modulation of alloreactive T cell responses has been a main strategy to reduce GVHD (Choi et al., Blood, 2010, 116:129-139; Bleakley and Riddell, Immunol Cell Biol, 2011, 89:396-407; Blazar et al., Nat Rev Immunol, 2012, 12:443-458; Shlomchik, Nat Rev Immunol, 2007, 7:340-352).

Interestingly, induction of alloreactive T cells does not necessarily lead to GVHD. Effector memory T cells (TEM), which were derived from non-alloantigen-sensitized mice, are unable to mediate GVHD (Anderson et al., J Clin Invest, 2003, 112:101-108; Chen et al., Blood, 2004, 103:1534-1541). Although these cells responded to alloantigen and mediated graft-versus-leukemia (GVL) effect, they showed impaired expansion in local tissues (Anderson et al., J Clin Invest, 2003, 112:101-108; Chen et al., Blood, 2004, 103:1534-1541; Zheng et al., Blood, 2008, 111:2476-2484; Chen et al., Blood, 2007, 109:3115-3123). This non-sensitized TEM pool might have less diverse T cell receptor (TCR) repertoire than the naïve T cell (TN) pool (Chen et al., Blood, 2004, 103:1534-1541), however, even host antigen-sensitized TEM showed reduced ability to trigger GVHD (Zhang et al., Biol Blood Marrow Transplant, 2012, 18:1488-1499; Juchem et al., Blood, 2011, 118:6209-6219). These host-reactive T cells responded to the antigen but died faster than TN, suggesting cell-intrinsic properties independent of TCR repertoire account for decreased ability of TEM to mediate GVHD (Juchem et al., Blood, 2011, 118:6209-6219). Thus, induction of qualitative changes in donor T cells can reduce their anti-host toxicities (Mochizuki et al., Immunotherapy, 2011, 3:1353-1366; Mochizuki et al., J Immunol, 2013, 190:3772-3782; Tran et al., J Clin Invest, 2013, 123:1590-604; Zhang et al., Blood, 2011, 117:299-308; Backer et al., Nat Immunol, 2014, 15:1143-1151).

Notch signaling is critical for alloreactive T cell responses. Notch receptors interact with Notch ligands of the δ-like and Jagged families (Kopan and Ilagan, Cell, 2009, 137:216-233; Maillard et al., Annu Rev Immunol, 2005, 23:945-974; Radtke et al., Immunity, 2010, 32:14-27), triggering the release of intracellular Notch (ICN) that activates Notch target genes (Kopan and Ilagan, Cell, 2009, 137:216-233; Maillard et al., Annu Rev Immunol, 2005, 23:945-974; Radtke et al., Immunity, 2010, 32:14-27). Inhibiting pan-Notch signaling in donor T cells reduced their production of IFN-γ and IL-17 (Zhang et al., Blood, 2011, 117:299-308). Notch ligand DLL4 mediates a dominant role for activating Notch signaling in alloreactive T cells (Tran et al., J Clin Invest, 2013, 123:1590-604). Inflammatory dendritic cells (DCs) that express high levels of DLL4 (named DLL4^{hi}DCs) occurred in HSCT mice early during GVHD induction and had a greater ability than DLL4-negative DCs to induce IFN-γ and IL-17 in alloantigen-activated T cells (Mochizuki et al., J Immunol, 2013, 190:3772-3782). Differentiated effector T cells have reportedly reduced capacity to proliferate and persist in vivo (Restifo et al., Nat Rev Immunol, 2012, 12:269-281; Kaech and Cui, Nat Rev Immunol, 2012, 12:749-761; Wherry et al., Nat Immunol, 2003, 4:225-234; Berner et al., Nat Med, 2007, 13:354-360). Without wishing to be bound by any particular theory, it is believed that in vitro priming with DLL4^{hi}DCs could allow the induction of alloreactive effector T cells with reduced GVHD toxicity.

The materials and methods employed in these experiments are now described.

### Materials and Methods

### Mice

C57BL/6 (B6, H-2^{b}), BALB/c (H-2^{d}), and B6xDBA/2 F1 (BDF1, H-2^{b/d}) mice were from Taconic (Rockville, Md.). Ifng-deficient (Ifng^{-/-}) mice and C3H.SW mice were purchased from The Jackson Laboratory. B6 mice conditionally expressing dominant-negative Mastermind-like 1 (DNMAML) were crossed to Cd4-Cre transgenic mice (Maillard et al., Blood, 2004, 104:1696-1702; Tu et al., J Exp Med, 2005, 202:1037-1042). Experimental protocols were approved by the University of Michigan's and Temple University's Committee on Use and Care of Animals.

### DC Production

For induction of DLL4^{hi}DCs, mouse bone marrow (BM) were cultured in RPMI1640 medium containing 10% FBS and recombinant mouse Flt3L (50 ng/ml). Eight days later, CD11 c⁺ immature DCs were magnetically sorted and incubated with lipopolysaccharide (LPS, 100 ng/ml) and R848 (resiquimod, 100 ng/ml), which activate TLR4 and TLR7/8 signaling, respectively (Hemmi et al., Nat Immunol, 2002, 3:196-200). Cells were harvested at day 9 for experiments. BM DCs produced from GM-CSF-based cultures were also stimulated with LPS+R848 and named GM-DCs.

### GVHD and Graft-Versus-Leukemia Response (GVL)

Mice underwent BMT as described (Zhang et al., Nat Med, 2005, 11:1299-1305). GVHD score and severity were graded by clinical parameters and histopathological analysis (Cooke et al., Blood, 1996, 88:3230-3239; Anderson et al., Blood, 2005, 2227-2234). To induce GVHD, we irradiated BALB/c recipients for 800 cGray from a ¹³⁷Cs source followed by transplantation with donor B6 TCD-BM with or without B6 CD4⁺ T cells. GVHD in BDF1 mice was induced by transfer of donor B6 TCD-BM together with B6 CD4⁺ T cells plus CD8⁺ T cells (He et al., Blood, 2012, 119:1274-1282). In C3H.SW anti-B6 mouse model of GVHD directed against minor histocompatibility antigens (miHA), B6 recipients were irradiated using 1000 cGray from X-ray source, followed by transfer of C3H.SW TCD-BM with or without CH3.SW CD8⁺ T cells. In GVL experiments, A20^{TGL} cells (1×10⁶) were injected 2 hours before HSCT, which reflects the residual disease in human transplant recipients as previously described (Reddy et al., PNAS, 2004, 101:3921-3926; Reddy et al., Nat Med, 2005, 11:1244-1249). Leukemic growth was monitored using bioluminescence imaging (Zhang et al., Blood, 2011, 117:299-308). In some experiments, C1498 myeloid leukemic cells (1.5×10⁴/mouse) and mastocytoma P815 cells (2×10³/mouse) were used to induce leukemia and tumor.

### Antibodies (Abs), Flow Cytometry Analysis and Cell Lines

Neutralizing antibodies specific to mouse DLL1, DLL4, J1 and J2 were prepared as described (Choi et al., Nat Rev Clin Oncol, 2014, 11:536-46; Choi et al., Blood, 2010, 116:129-139). All other antibodies used for immunofluorescence staining were purchased from eBioscience (San Diego, Calif.), BioLegend (San Diego, Calif.), or BD Biosciences (San Jose, Calif.). Magnetic Microbead-conjugated Abs and streptavidin were purchased from Miltenyi-Biotech (Auburn, Calif.). Recombinant human IL-2, mouse GM-CSF, mouse SCF, and mouse IL-4 were purchased from R&D Systems (Minneapolis, Minn.). Recombinant mouse Flt3 ligand (Flt3L) was purchased from Shenandoah Biotech (Warwick, Pa.). Flow cytometry analyses were performed using CyAn (Beckmann Coulter) and Canto cytometer (Becton Dickinson).

### Cell Preparations

T cell-depleted bone marrow (TCD-BM) was prepared by depleting T cells with microbead¬conjugated anti-CD4/CD8 antibodies (Bleakley and Riddell, Immunol Cell Biol, 2011). CD4⁺ and CD8⁺ naïve T cells (TN) were isolated from spleens and lymph nodes using microbead-conjugated antibodies (MiniMACS; Miltenyi Biotech), followed by depletion of CD44-positive cells. Purity was consistently >92%. The preparation of lamina propria lymphocytes (LPL) and intra epithelial lymphocytes (IEL) was performed as previously described (Blazar et al., Nat Rev Immunol, 2012, 12:443-458).

### Real-Time RT-PCR

Total RNA was extracted from sorted DCs and CD4⁺ T cells using TRIzol (Invitrogen Life Technologies). cDNA was quantified through quantitative real-time polymerase chain reaction (PCR) using a SYBR Green PCR mix on a Mastercycler realplex (Eppendorf). Thermocycler conditions included an initial holding at 95° C. for 2 min; this was followed by a three-step PCR program, as follows: 95° C. for 30 s, 55° C. for 30 s, and 72° C. for 30 s for 40 cycles. Transcript abundance was calculated using the ΔΔCt method (normalization with GAPDH or 18S).

### Mixed Lymphocyte Reaction (MLR) and Cytotoxicity Assay

Donor B6-derived CD4⁺ or CD8⁺ TN cells were stimulated with in vitro cultured DCs from B6, BALB/c or BDF1 mice in 96-well U-bottom plates in complete medium. In C3H.SW anti-B6 mouse model, donor C3H.SW-derived CD8⁺ T cells were stimulated with B6 derived DLL4hi DCs. The ratio of DCs and CD4⁺ TN is 1:4, unless indicated otherwise. Cells were cultured for five days prior to in vivo injection or to assess surface antigens, CF SE dilution and intracellular cytokine production as described (Bleakley and Riddell, Immunol Cell Biol, 2011, 89:396-407). In vitro cytotoxicity assay against A20 leukemic cells was performed as previously described (Blazar et al., Nat Rev Immunol, 2012, 12:443-458).

### Statistical Analysis

Survival in different groups was compared using the log-rank test. Comparison of means was done using the 2-tailed unpaired Student's t test.

The results employed in these experiments are now described.

### Generation of Murine DLL4hiDCs

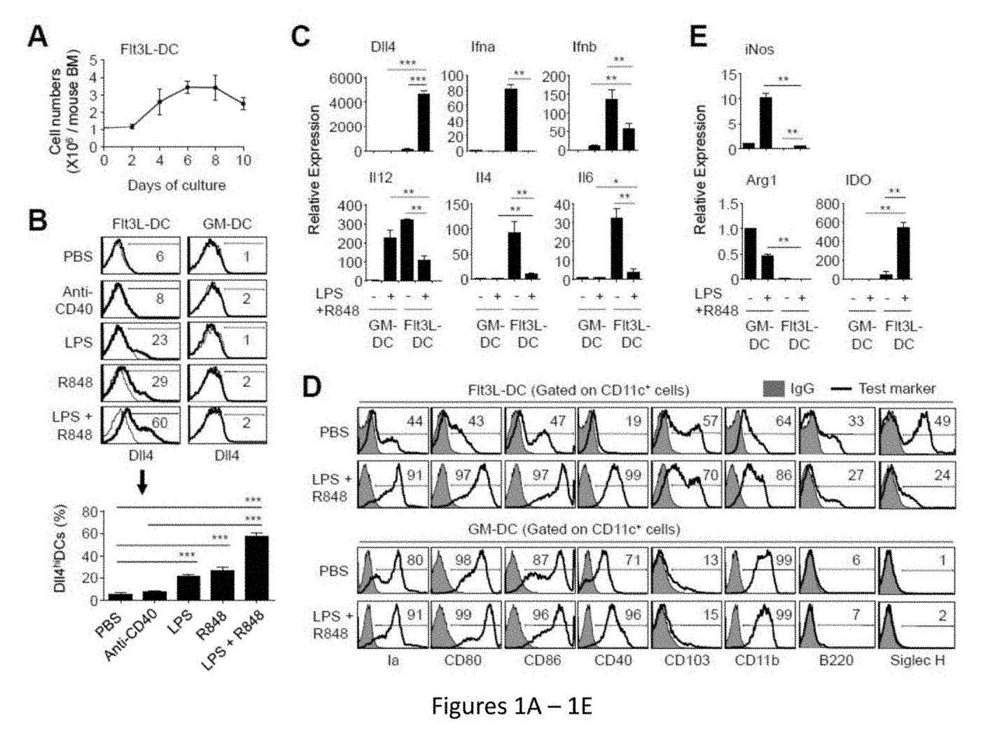

Only about 5% of DCs derived from normal mice express DLL4 (Mochizuki et al., J Immunol, 2013, 190:3772-3782). To provide adequate numbers of DLL4^{hi}DCs for therapeutic translation, a culture system capable of generating sufficient numbers of DLL4^{hi}DCs was developed. Our previously identified phenotypic similarities between DLL4^{hi}DCs and plasmacytoid DCs (pDCs) (Mochizuki et al., J Immunol, 2013, 190:3772-3782), the latter of which are induced in vitro using Flt3L (Gilliet et al., J Exp Med, 2002, 195:953-958). To generate DLL4^{hi}DCs, murine BM were cultured with Flt3L. Flt3L induced CD11c+ DCs (named Flt3L-DCs) that were DLL4 negative (FIG. 1A,B). Overnight incubation with LPS, R848 or LPS+R848 induced DLL4 expression on the surface of Flt3L-DCs. Concurrent stimulation with LPS and R848 induced the highest level of DLL4 (FIG. 1B) and was therefore used for all subsequent experiments. These DLL4-expressing Flt3L-DCs are henceforth referred to as DLL4^{hi}DCs. Averagely 2.5×10⁶ DLL4^{hi}DCs were generated in cultures from one mouse BM, and more than 60% of them expressed high levels of DLL4.

Flt3L-DCs stimulated with LPS and R848 showed a dramatic decrease in mRNA transcripts encoding inflammatory cytokines (e.g., Ifna, Ifnb, Il2, Il4 and Il6) (FIG. 1C), but simultaneously upregulated molecules associated with DC maturation, including antigen-presenting molecules (Ia), costimulatory molecules (CD80, CD86 and CD40), CD103 (marker of migratory DCs) and CD11b (marker for conventional DCs) (Steinman and Banchereau, Nature, 2007, 449:419-426) (FIG. 1D). Thus, induction of DLL4 on Flt3L-DCs is associated with maturation. Immature DLL4DCs expressed B220 (33%) and Siglec H (49%) (FIG. 1D), which are pDC markers (Zhang et al., Blood, 2006, 107:3600-3608), suggesting that DLL4^{hi}DCs may be originated from both pDCs and cDCs. In contrast, GM-DCs did not express DLL4 despite stimulation with LPS+R848 (FIG. 1B and FIG. 1C). As compared to DLL4^{hi}DCs, GM-DCs had lower levels of Ifngb, Il4, Il6 and Ido, expressed higher levels of iNOS and Arg1 (arginase I) and expressed no surface CD103 (FIGS. 1C to FIG. 1E).

### DLL4hiDC-Induced Alloreactive T Cells have Reduced Ability to Cause GVHD

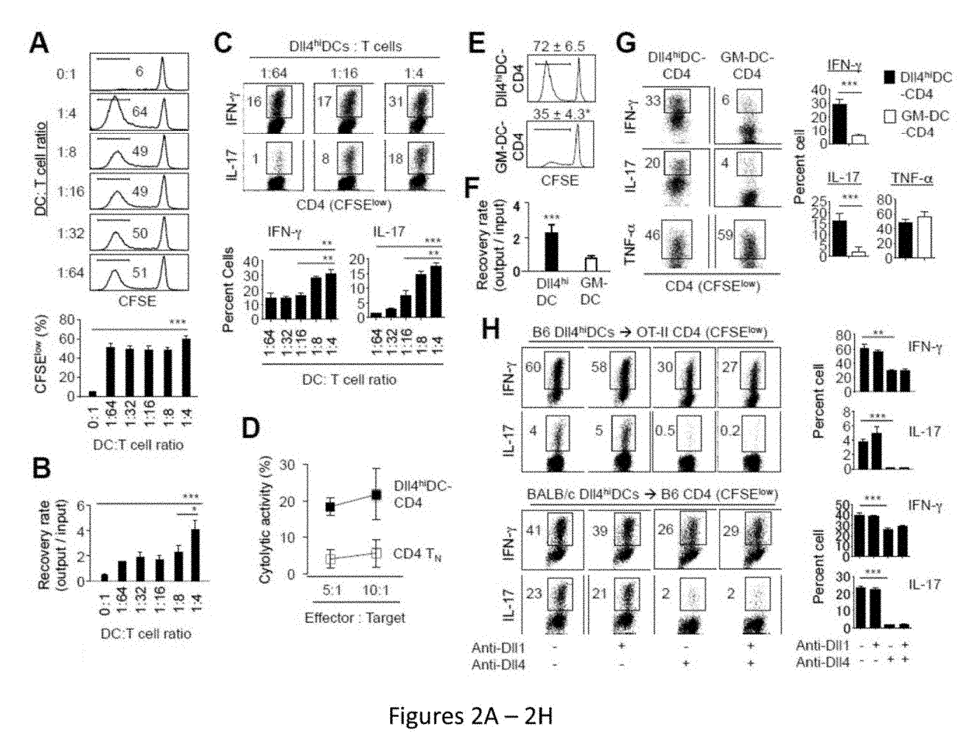

To determine if donor CD4⁺ TN could be programmed by DLL4^{hi}DCs for reducing GVHD, B6 CD4⁺ TN were incubated with or without escalating numbers of BALB/c DLL4^{hi}DCs for 5 days. These DLL4^{hi}DC-activated CD4⁺ T cells underwent extensive proliferation, as evidenced by low levels of CFSE (CFSE^{low}) (FIG. 2A). As a result, DLL4^{hi}DCs induced expansion of donor CD4⁺ T cells (FIG. 2B), production of high levels of IFN-γ and IL-17 (FIG. 2C). These DLL4^{hi}DC-induced T cells were able to kill A20 leukemia cells in vitro (FIG. 2D). As compared to DLL4^{hi}DCs, GM-DCs were significantly less potent in promoting proliferation of allogeneic CD4⁺ T cells and their production of IFN-γ and IL-17 (FIG. 2E to FIG. 2G), confirming again the functional difference between DLL4^{hi}DCs and GM-DCs.

### Allogeneic MLR Activates Polyclonal T Cells and is unable to Model Alloreactivity to a Single Alloantigen

CD4⁺ TN specific to OT-II peptide (OVA232-239) were isolated from TCR transgenic OT-II mice and cultured with syngeneic DLL4^{hi}DCs pulsed by OT-II peptides. Addition of DLL4^{hi}DCs and OT-II peptides induced vigorous proliferation of OT-II CD4⁺ TN (Figure S1) and production of IFN-γ and IL-17 (FIG. 2H). Blocking DLL4 but not DLL1 using neutralizing Abs reduced IFN-γ and IL-17 by CD4⁺ T cells (FIG. 2H). DLL4 blockade also inhibited IFN-γ- and IL-17-production by B6 CD4⁺ TN stimulated with BALB/c DLL4^{hi}DCs (FIG. 2H). These findings demonstrate that antigenic peptides presented by DLL4^{hi}DCs elicit specific T cell responses and DLL4 is critical for promoting IFN-γ- and IL-17-production.

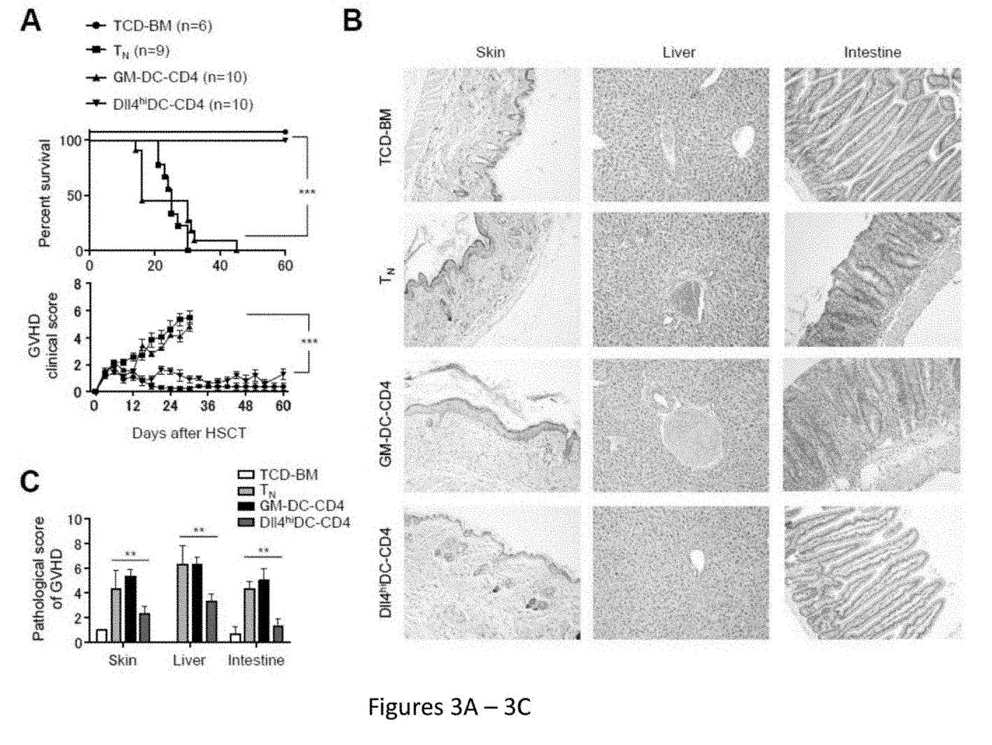

To test the ability of allogeneic DLL4^{hi}DC-induced CD4⁺ T cells (DLL4^{hi}DC-CD4) to mediate GVHD, primed T cells were harvested 5 days after stimulation and transferred together with TCD-BM into lethally irradiated BALB/c mice, with unstimulated CD4⁺ TN as controls. Transfer of donor CD4⁺ TN caused lethal GVHD (FIG. 3A to FIG. 3C). In contrast, BALB/c mice receiving DLL4^{hi}DC-CD4⁺ T cells developed only minimal GVHD and complete survival (FIG. 3A to FIG. 3C). Histological examination demonstrated that DLL4^{hi}DC-CD4⁺ T cells caused less severe tissue inflammation compared to CD4⁺ TN (FIG. 3B and FIG. 3C). Notably, GM-DC-CD4⁺ T cells mediated severe GVHD, with all recipients dying from the disease (FIG. 3A to FIG. 3C). These results indicate that DLL4^{hi}DCs rather than GM-DCs can be used for reducing GVHD toxicity of donor CD4⁺ TN.

### DLL4^{hi}DC-CD4⁺ T Cells Retain Anti-Leukemia Activity

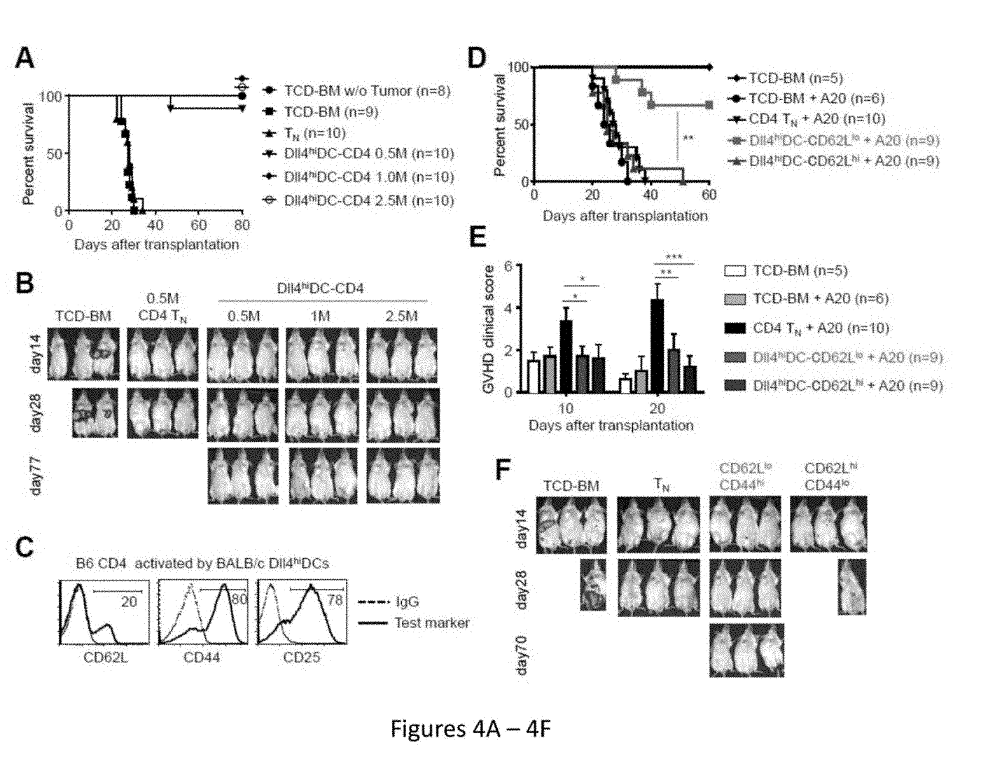

To determine whether these DLL4^{hi}DC-CD4⁺ T cells preserved GVL activity, B6 TCD-BM were transplanted with or without titrated numbers of DLL4^{hi}DC-CD4⁺ T cells (ranging from 0.5×10⁶ to 2.5×10⁶) into lethally irradiated BALB/c mice. A20TGL leukemia cells were injected into these recipients to induce leukemia. Mice receiving TCD-BM died from leukemia, while mice receiving TCD-BM plus CD4⁺ TN (0.5×10⁶ cells/mouse) eliminated leukemia cells but died from GVHD (FIG. 4A,B). In contrast, transfer of up to 2.5×10⁶ T cells did not induce severe GVHD but protected recipients against leukemic cell challenge (FIG. 4A,B). This suggests that our strategy dramatically expands the safety range of infused donor T cell dose by at least 5-fold.

CD4⁺ T cells from DLL4^{hi}DC-induced cultures contained at least two different subsets: CD44^{hi}CD62L^{lo} effector T cells (80%) and CD44^{lo}CD62L^{hi} TN-like cells (20%) (FIG. 4C). TN have a greater ability than effector T cells to proliferate and survive after adoptive transfer (Restifo et al., Nat Rev Immunol, 2012, 12:269-281; Zhang et al., Nat Med, 2005, 11:1299-1305; Zhang et al., Blood, 2004, 103:3970-3978). To exclude if these CD44^{lo}CD62L^{hi} TN-like cells might account for eliminating leukemic cells in allogeneic HSCT mice, DLL4^{hi}DC-induced T cells were flow-sorted into two subsets: CD44^{hi}CD62L^{lo} cells and CD44^{lo}CD62L^{hi} cells (Figure S2). Transfer of either CD44^{hi}CD62L^{lo} cells or CD44^{lo}CD62L^{hi} cells did not cause severe GVHD in these recipients (FIG. 4D,E). Interestingly, all mice receiving these CD44^{lo}CD62L^{hi} TN-like cells died from leukemia, whereas six of nine mice receiving DLL4^{hi}DC-induced CD44^{hi}CD62L^{lo} cells survived without leukemia and GVHD (FIG. 4D-F). Thus, allogeneic DLL4^{hi}DC-induced CD44^{hi}CD62L^{lo} effector T cells are responsible for controlling leukemia. In contrast, CD44^{lo} CD62L^{hi} cells that survived in DLL4^{hi}DC cultures might contain alloreactive T cells lower than the threshold sufficient to mediating GVHD. Indeed, transfer of higher dose of CD44^{lo}CD62L^{hi} cells (i.e., 2.5×10⁶) caused lethal GVHD.

Upon BALB/c GM-DC activation, B6 CD4⁺ T cells upregulated CD25 but decreased CD62L, generating both CD62L^{hi} and CD62L^{lo} cell subsets (Figure S3A). GM-DC-induced CD62L CD4⁺ T cells were unable to mediate either GVHD or GVL, whereas transfer of GM-DC-induced CD62L^{lo} CD4⁺ T cells caused GVHD (Figure S3B¬D), confirming that GM-DCs are not useful for reducing the capacity of donor CD4⁺ TN to mediate GVHD.

### DLL4^{hi}DC Stimulation Reduces Capacity of CD8⁺ T Cells to Mediate GVHD

To rule out the possibility of a model-specific effect, a B6 anti-BDF1 mouse model was used to examine the GVH effect of DLL4hiDC-induced T cells. As compared to DLL4^{hi}DC-CD4⁺ T cells, DLL4^{hi}DC-CD8⁺ T cells produced higher levels of IFN-γ (Figure S4A). Both DLL4^{hi}DC-CD4⁺ T cells and DLL4^{hi}DC-CD8⁺ T cells were transplanted, together with B6 TCD-BM, into irradiated BDF1 mice that had been inoculated with mastocytoma P815 cells 2 hours earlier. Macroscopic examination of tumor in the lung and liver was performed to monitor tumor growth. Transfer of unstimulated T cells caused severe GVHD, with 5/8 mice dying from GVHD and 3/8 dying with tumor (Figure S4B-C). In contrast, BDF1 mice receiving DLL4^{hi}DC-induced T cells did not develop GVHD (Figure S4B-D). These DLL4^{hi}DC-induced T cells retained anti-tumor activities evidenced by significantly prolonged median survival time compared to mice receiving TCD-BM plus P815 cells (36 days vs. 11 days, Figure S4B-C).

To determine the precise impact of DLL4^{hi}DCs on CD8⁺ T cell-mediated GVHD directed against miHAs, DLL4^{hi}DCs were generated from B6 mouse BM and added into cultures of C3H.SW CD8⁺ TN. Five days later, DLL4^{hi}DC-CD8⁺ T cells vigorously proliferated and produced high levels of IFN-γ and TNF-α, which were inhibited upon DLL4 blockade (Figure S5A,B). Transfer of DLL4^{hi}DC-CD8⁺ T cells caused significantly less severe GVHD in B6 mice than did CD8⁺ TN, as evidenced by improved overall survival (60% versus 10%, respectively) and reduced clinical scores (Figure S5C,D). Transfer of DLL4^{hi}DC-CD8⁺ T cells markedly prolonged the mean survival time of B6 mice inoculated with C1498 leukemic cells compared to TCD¬BM (40 days vs. 23 days, Figure S5E,F). These data reveal a specific effect of DLL4 on the alloreactive CD8⁺ T cell responses, and suggest that DLL4^{hi}DCs can be used for reducing GVHD toxicity mediated by CD8⁺ T cells.

### DLL4^{hi}DC-CD4⁺ T Cells have Impaired Capacity to Expand in Vivo

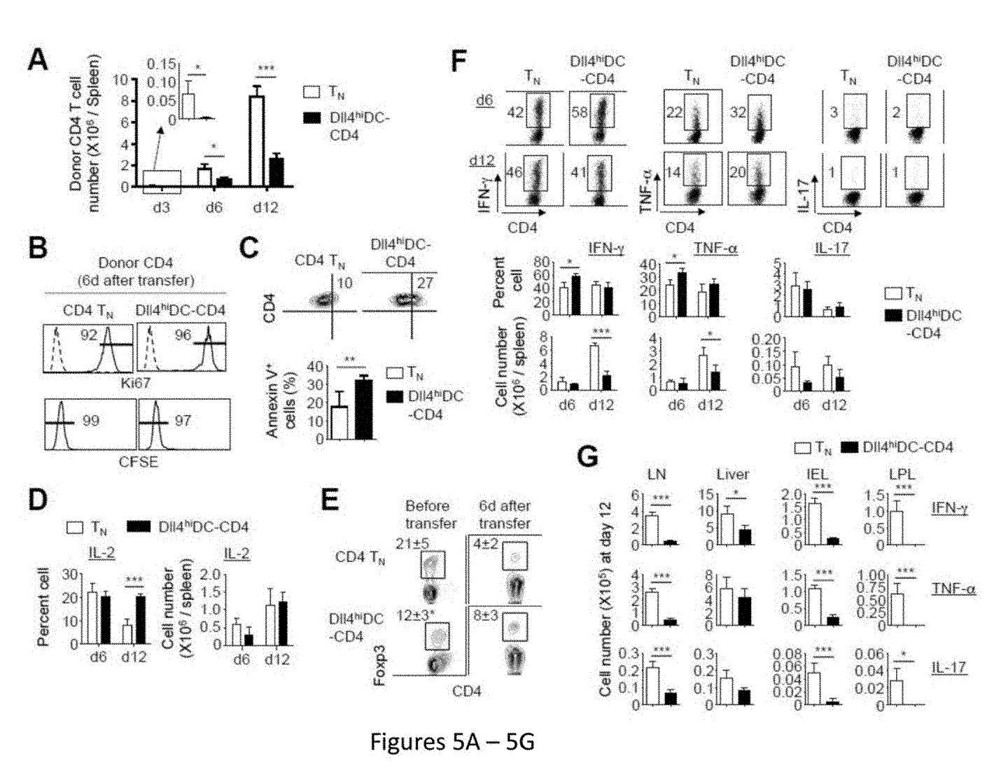

DLL4^{hi}DC-CD4⁺ T cells and CD4⁺ TN were transferred into irradiated allogeneic BALB/c mice. As compared to TN recipients, DLL4^{hi}DC-CD4⁺ T cell recipients showed significantly fewer donor T cells in the spleen at day 3, day 6 and day 12 (FIG. 5A). Transferred DLL4^{hi}DC-CD4⁺ T cells had undergone similar proliferation as measured by Ki67 and CFSE dilution 6 days after transplantation (FIG. 5B), but had a 1.5-fold higher frequency of apoptotic cells (FIG. 5C). Anergic T cells have decreased capacity to produce IL-2 and might contribute to impaired expansion of alloreactive T cells (Norton et al., J Immunol, 1991, 146:1125-1129). DLL4^{hi}DC-CD4⁺ T cells produced higher levels of IL-2 than CD4⁺ TN (FIG. 5D). FoxP3⁺ regulatory T cells (Treg) also affect GVH reactions (Blazar et al., Nat Rev Immunol, 2012, 12:443-458; Nguyen et al., Blood, 2007, 109:2649-2656). As compared to CD4⁺ TN, DLL4^{hi}DC-CD4⁺ T cells contained fewer number of Treg prior to transfer but showed slightly higher frequency of Treg without statistical significance 6 days after transfer (FIG. 5E). These data suggest that impaired expansion of DLL4^{hi}DC-T cells in vivo may be primarily attributable to increased apoptosis.

Interestingly, DLL4^{hi}DC-CD4⁺ T cells produced higher levels of IFN-γ and TNF-α than CD4⁺ TN cells 6 days after transfer (FIG. 5F). Twelve days after transplantation, DLL4^{hi}DC-CD4⁺ T cells and CD4⁺ TN produced similar levels of IFN-γ and TNF-α (FIG. 5F). However, the impaired expansion of transferred DLL4^{hi}DC-CD4⁺ T cells led to an overall reduction of IFN-γ- and TNF-α-producing alloreactive T cells in the spleen, LN, liver and intestine compared to CD4 TN (FIG. 5F,G).

Whether Th17 cells mediate GVHD remains controversial (Carlson et al., Blood, 2009, 113:1365-1374; Yi et al., Blood, 2009, 114:3101-3112; Kappel et al., Blood, 2009, 113:945-952). Th17 cells were shown to induce GVHD but donor T cells lacking IL-17 induce worse GVHD when compared with normal T cells (Yi et al., Blood, 2009, 114:3101-3112). Interestingly, although DLL4^{hi}DC-CD4⁺ T cells produced high levels of IL-17 prior to their transplantation, they did not sustain this capacity following transfer (FIG. 5F,G). Th17 cells have a demonstrated plasticity and have been shown to transition to Th1 cells (Muranski et al., Immunity, 2011, 35:972-985). Thus, whether the decrease in production of IL-17 seen in DLL4^{hi}DC-CD4⁺ T cells in vivo results from impaired survival and/or IFN-γ-mediated repression of IL-17 during GVH reaction seen by other studies (Lu et al., Blood, 2012, 119:1075-1085), has yet to be determined. Collectively, the data suggest that impaired expansion capability of DLL4^{hi}DC-CD4⁺ T cells in GVHD target organs may account for protection against GVHD.

### T Cell IFN-γ is Required for DLL4^{hi}DC Programming of T Cell Alloreactivity

Previous studies suggested that administration of IFN-γ to allogeneic HSCT recipients at the time of transplantation repressed GVHD (Brok et al., J Immunol, 1993, 151:6451-6459). T cells from Ifng^{-/-} donors induced more severe systemic GVHD than WT donors (Yi et al., Blood, 2009, 114:3101-3112, Wang et al., Blood, 2009, 113:3612-3619; Yang et al., Blood, 2002, 99:4207-4215; Yang et al., J Cin Invest, 1998, 102:2126-2135; Burman et al., Blood, 2007, 110:1064-1072). To determine whether IFN-γ was required for reducing the capacity of DLL4^{hi}DC-induced CD4⁺ T cells to mediate GVHD, Ifng^{-/-} B6 CD4⁺ T cells were cultured with BALB/c DLL4^{hi}DCs. Ifng^{-/-} CD4⁺ T cells did not produce IFN-γ, had similar levels of IL-4 and TNF-α, and produced more IL-17 (Figure S6). Thus, Ifng^{-/-} CD4⁺ T cells can respond to allogeneic DLL4^{hi}DCs.

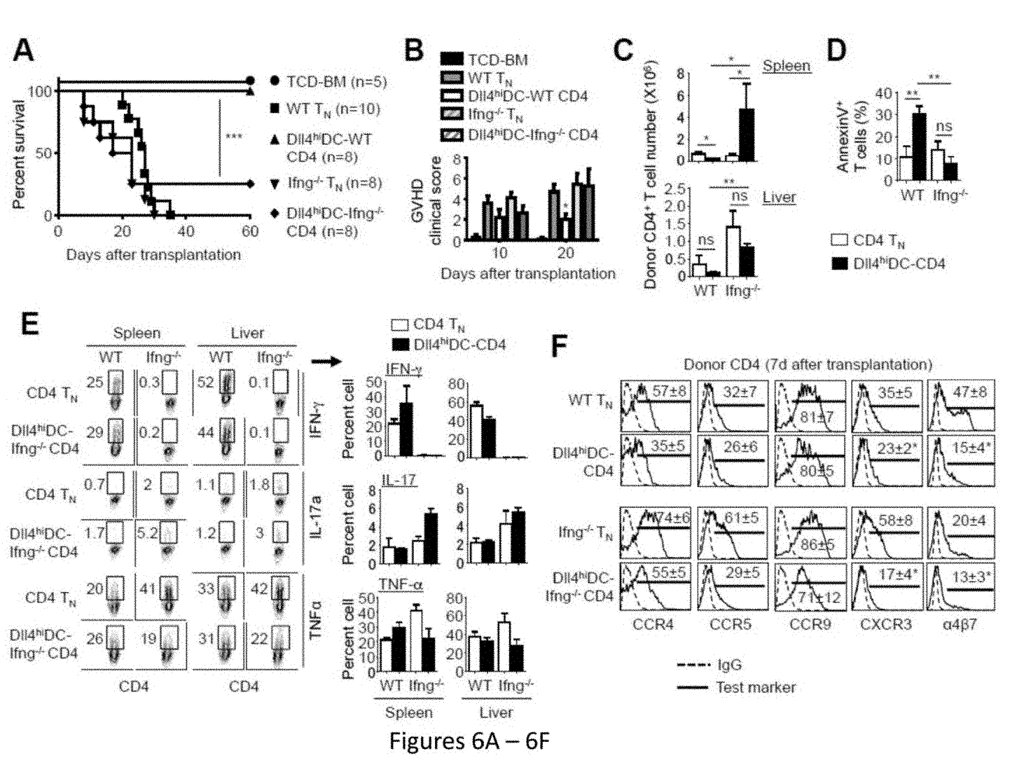

As expected, Ifng^{-/-} CD4⁺ TN induced lethal GVHD in all BALB/c recipients (FIG. 6A,B). Notably, DLL4^{hi}DC-Ifng^{-/-} CD4⁺ T cells also induced lethal GVHD in 6/8 of BALB/c recipients (FIG. 6A,B). Similar to WT CD4⁺ TN, both DLL4^{hu}DC-Ifng^{-/-} CD4⁺ T cells and Ifng^{-/-} CD4⁺ TN infiltrated the spleen and the liver, with 3-fold more donor T cells in the spleen of DLL4^{hi}DC-Ifng^{-/-} CD4⁺ T cell recipients 6 days after transfer (FIG. 6C). Loss of IFN-γ led to markedly decreased apoptosis of DLL4^{hi}DC¬Ifng^{-/-} CD4⁺ T cells compared to their WT counterparts (FIG. 6D). Despite DLL4^{hi}DC induction, Ifng^{-/-} CD4⁺ T cells retained the capacity to produce TNF-α and IL-17 during GVH reaction (FIG. 6E). These results indicate that IFN-γ is important for enhanced apoptotic death of DLL4^{hi}DC-WT CD4⁺ T cells and their inability to mediate GVHD.

The observation that mice receiving DLL4^{hi}DC-CD4⁺ T cells had relatively fewer donor T cells in the intestine compared to the spleen suggested the possibility of impaired migration (FIG. 5F,G). To test this, DLL4^{hi}DC-CD4⁺ T cell expression of chemokine receptors (e.g., CCR4, CCR5, CCR9 and CXCR3) and adhesion molecule integrin-α4β7 was assessed. These molecules are important for the migration of alloreactive T cells into GVHD target tissues, including skin, intestine and liver (Blazar et al., Nat Rev Immunol, 2012, 12:443-458; Wysocki et al., Blood, 2005, 105:4191-4199). As compared to donor WT CD4⁺ TN, donor DLL4^{hi}DC-CD4⁺ T cells had lower levels of α4β7, CCR4 and CXCR3 (FIG. 6F). Interestingly, Ifng^{-/-} CD4⁺ T cells, which are known to induce less severe GVHD in the gut than their WT counterparts (Burman et al., Blood, 2007, 110:1064-1072), also expressed lower percentage of α4β7 (FIG. 6F).

### DLL4^{hi}DCs Promote Effector Differentiation Via a Notch-Dependent Mechanism

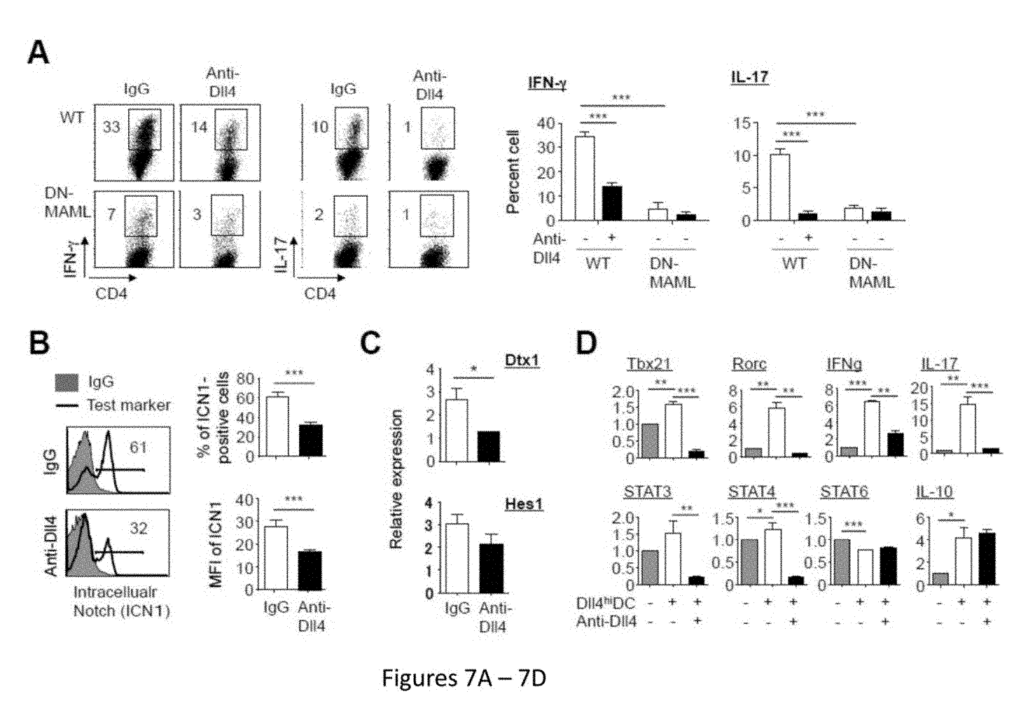

To determine the role of Notch in DLL4^{hi}DC induction of alloreactive T cells, T cells expressing DN-MAML, a specific pan-Notch inhibitor (Fang et al., Immunity, 2007, 27:100-110) were used. In the presence of allogeneic DLL4^{hi}DCs, DN-MAML CD4⁺ T cells produced 5-times fewer Th1 cells in cultures compared to WT CD4⁺ T cells (FIG. 7A). Addition of anti-DLL4 Ab did not further decrease Th1 cells in cultures of DN-MAML CD4⁺ T cells (FIG. 7A), suggesting the importance of Notch signaling in DLL4^{hi}DC-mediated Th1 differentiation.

DLL4 is a known activator of Notch1 signaling (Tran et al., J Clin Invest, 2013, 123:1590-604; Radtke et al., Immunity, 2010, 32:14-27), therefore it was hypothesized that DLL4^{hi}DC activation induced ICN1 in alloreactive T cells. Indeed, 61% of DLL4^{hi}DC¬CD4⁺ T cells produced high levels of ICNs. Blocking DLL4 led to significant reduction of ICN1 (FIG. 7B) and its target genes (e.g., Dtx1and Hes1) in WT CD4⁺ T cells stimulated with DLL4^{hi}DCs (FIG. 7C), indicating the activation of Notch1 in these T cells. Transcription factors T-bet and Stat4 are essential for inducing Ifng transcription in Th1 cells (Zhu et al., Annu Rev Immunol, 2010, 28:445-489). Addition of anti-DLL4 Ab reduced the expression of Ifng, Tbx21 and Stat4 in CD4⁺ T cells activated by allogeneic DLL4^{hi}DCs (FIG. 7D). Thus, DLL4 activation of Notch signaling is important for the expression of transcription factors critical for Th1 differentiation.

This invention provides a novel cellular programming approach that produces large numbers of alloreactive effector T cells incapable of causing severe GVHD but retaining GVL effects. To facilitate this strategy, a platform was developed that produces large numbers of DLL4^{hi}DCs from murine BM. Upon in vitro stimulation by allogeneic DLL4^{hi}DCs, donor CD4⁺ TN became alloreactive effector T cells that produced high levels of IFN-γ and IL-17. Adoptive transfer of these DLL4^{hi}DC-induced T cells eliminated leukemic cells without causing severe GVHD, leading to significantly improved survival of leukemic mice undergoing allogeneic HSCT. This strategy has several potential advantages compared to current and developing methods for the modification of donor T cell grafts to reduce GVHD, including a relatively short period of culture, no requirement for T cell subset selection and dramatically expanded safety range of infused donor T cell dose. Collectively, these findings demonstrate that DLL4^{hi}DC programming can overcome GVHD toxicity of donor T cells and produce leukemia-reactive T cells for improving immunotherapy.

Without being bound by a particular theory, the reduction of DLL4^{hi}DC-induced T cells in mediating GVHD may be explained by their impaired expansion capability in vivo. DLL4^{hi}DC-induced CD4⁺ T cells underwent enhanced apoptosis early after transfer compared to unstimulated CD4⁺ TN, leading to an overall reduction of donor T cells in GVHD target organs. Effector differentiation is known to be associated with reduced proliferation and expansion of antigen-specific T cells. Likewise, the intrinsic defect of antigen-sensitized CD4⁺ TEM in mediating GVHD was linked to their limited expansion in target tissues. However, the underlying molecular mechanisms remain largely unknown.

IFN-γ plays paradoxical roles in regulating GVH reactivity against lymphohematopoietic cells versus non-hematopoietic tissues (Yi et al., Blood, 2009, 114:3101-3112, Wang et al., Blood, 2009, 113:3612-3619; Yang et al., Blood, 2002, 99:4207-4215; Yang et al., J Cin Invest, 1998, 102:2126-2135; Burman et al., Blood, 2007, 110:1064-1072). IFN-γ prevented the development of severe GVHD early after allogeneic HSCT in mice, and repressed the damage to the skin and lung (Wang et al., Blood, 2009, 113:3612-3619; Yang et al., Blood, 2002, 99:4207-4215; Yang et al., J Cin Invest, 1998, 102:2126-2135). IFN-γ upregulated expression of the immune check-point inhibitor PD-L1 in lung parenchyma, leading to reduced expansion of alloreactive T cells and protection against lung GVHD (Yi et al., Blood, 2009, 114:3101-3112. IFN-γ had been implicated in down-regulation of T cell immune responses with signaling inducing apoptosis in activated T cells (Berner et al., Nat Med, 2007, 13:354-360; Wang et al., Blood, 2009, 113:3612-3619; Yang et al., Blood, 2002, 99:4207-4215; Robb and Hill, Blood, 2012, 119:5351-5358). However, IFN-γ has also been implicated in mediating GVH reactions against hematopoietic tissues (Wang et al., Blood, 2009, 113:3612-3619; Yang et al., Blood, 2002, 99:4207-4215) and gastrointestinal tract (Burman et al., Blood, 2007, 110:1064-1072). As compared to unstimulated Ifng^{-/-} CD4⁺ TN, DLL4^{hi}DC-induced Ifng^{-/-} CD4⁺ T cells had greater ability to expand in the spleen, exhibited similar capacity to accumulate and survive in the liver, and caused severe GVHD after transplantation. These data suggest that T cell IFN-γ is required to limit the expansion of DLL4^{hi}DC-induced T cells in GVHD target tissues and development of GVHD. Further studies will investigate the mechanism by which DLL4^{hi}DC-CD4⁺ T cells produce high levels of IFN-γ but have reduced ability to accumulate and expand in the gut.

Notch simultaneously regulates the genetic programs important for effector differentiation independently of cytokine signals (Bailis et al., Immunity 2013, 39:148-159; Keerthivasan et al., J Immunol, 2011, 187:692-701; Mukherje et al., J Immunol, 2009, 182:7381-7388). Notch directly binds to the regulatory regions of genes encoding transcription factors (e.g., Tbx21, Rorc, Gata3) and cytokines (e.g., Ifng, Il17 and Il4) (Fang et al., Immunity, 2007, 27:110-110; Bailis et al., Immunity 2013, 39:148-159; Keerthivasan et al., J Immunol, 2011, 187:692-701; Mukherje et al., J Immunol, 2009, 182:7381-7388), thereby amplifying signals for Th differentiation (Bailis et al., Immunity 2013, 39:148-159). DLL4^{hi}DC-derived DLL4 is critical for activation of Notch signaling in alloreactive T cells. Inhibition of DLL4 led to dramatically reduced expression of Tbx21 in CD4⁺ T cells stimulated by allogeneic DLL4^{hi}DCs. These results are in agreement with previous observations that the intracellular Notch1 binds to the regulatory regions of Tbx21 (Bailis et al., Immunity 2013, 39:148-159; Skokos and Nussenzweig, J Exp Med, 2007, 204:1525-1531; Roderick et al., J Exp Med, 2013, 210:1311-1329). Blocking DLL4 led to marked reduction of ICN1 in CD4⁺ T cells activated by DLL4^{hi}DCs. Inhibiting DLL4 also caused significant reduction of Stat4 in DLL4^{hi}DC¬induced CD4⁺ T cells. These data suggest that DLL4^{hi}DCs and their derived DLL4 may regulate Th1 cell differentiation through Notch-dependent pathway.

The differential effects of GM-DCs and DLL4^{hi}DCs on eliciting alloreactive T cell responses may involve a complex mechanism. First, activation of T cell Notch signaling may explain the difference in reducing the ability of alloreactive effector T cells to mediate GVHD between GM-DCs and DLL4^{hi}DCs. DLL4^{hi}DC-induced T cells have undergone greater extent of proliferation and effector differentiation than GM¬DC-induced T cells. This is in agreement with that Notch signaling has broad impact on promoting the production of multiple lineages of alloreactive effector T cells. Since IFN-γ produced by DLL4^{hi}DC-CD4⁺ T cells was critical for limiting their expansion in GVHD target tissues, the inferior capacity of GM-DCs to DLL4^{hi}DCs to induce IFN-γ-producing T cells could account for the difference in reducing T cell-mediated GVHD between these two distinct types of DCs. Second, GM-DCs and DLL4^{hi}DCs displayed significant differences in expression of genes associated with inflammatory cytokines (e.g., Ifna, Ifnb, Il4, Il6) and immune modulatory molecules (e.g., iNos, Arg1 and Ido). Since these molecules are reportedly important for regulating T cell immune responses, they might have significant impact on DC-programming of alloreactive T cell responses.

DLL4^{hi}DC-induced alloreactive effector T cells had acquired the capable of killing leukemic cells. This may potentially improve the anti-leukemic response early after HSCT, and overcome some barriers to the GVL response such as high disease burden and pharmacologic immunosuppression. Importantly, these DLL4^{hi}DC-induced T cells continually produced high levels of effector cytokines such as IFN-γ and TNF-α in vivo after transfer, though they had limited expansion capacity in GVHD target organs. In addition, DLL4^{hi}DC-induced T cells elicited GVL activity with a wider safety range of infused T cell numbers without causing GVHD than their naïve counterparts. DLL4^{hi}DCs loaded with leukemia-associated antigens may facilitate the selection and expansion of leukemic cell-reactive T cells that mediate anti-leukemia activity.

Given the potent effects of DLL4^{hi}DCs and DLL4 on eliciting Th1- and Th17-cell responses in vitro, DLL4^{hi}DCs can be used as a platform to identify T cell clones selectively reactive to leukemic associated antigens.

### Example 2

### DLL4-Engineered Antigen-Presenting Cells and Tumor Vaccine

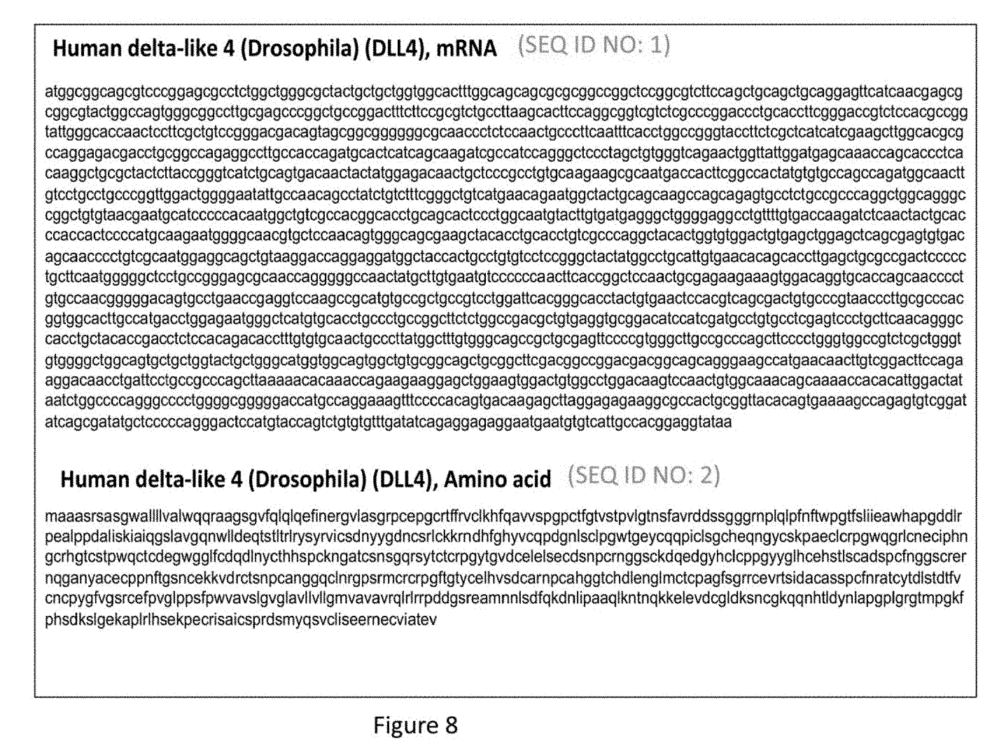

To provide sufficient numbers of human DLL4 DCs for in vitro priming, lentivirus encoding human DLL4 was made and used to infect human monocyte-derived DCs (Mo-DC) and produce DLL4 Mo-DCs. The mRNA and protein sequence of human DLL4 are shown in FIG. 8.

### Engineering Mo-DC Using Lentivirus-Encoding DLL4

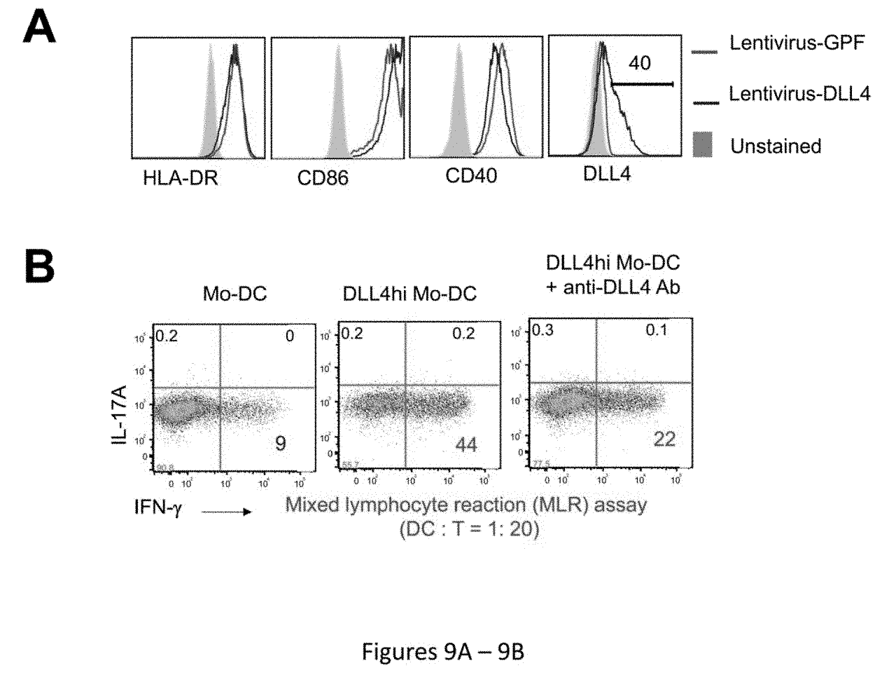

To provide sufficient numbers of human DLL4 DCs for in vitro priming, lentivirus encoding human DLL4 (SEQ ID NO:1) was made and used to infect human monocyte-derived DCs (Mo-DC). Viral introduction of DLL4 into these DCs produced DLL4^{hi} Mo-DCs, but did not alter their expression of HLD-DR and co-stimulatory molecules CD40 and CD86 (FIG. 9A). Interestingly, when added to allogeneic MLR cultures, these DLL4^{hi} Mo-DCs induced 4-fold more CD4 TH1 cells than control Mo-DCs (FIG. 9B). Blocking DLL4 by its specific Ab markedly reduced the effect of these DLL4^{hi} Mo-DCs on promoting TH1 cell response (FIG. 9B). These results clearly demonstrate the potent effect of DLL4hi DCs and DLL4 on eliciting human T cell responses.

### DLL4-Engineered Chimeric Antigen Receptor (CAR) T Cells Specific for CD19 (CAR-CD19 T Cells)

CAR-CD19 T cells are potentially able to cure patients with advanced B cell malignancies. However, a number of variables, including ex vivo culture conditions, poor in vivo effector functionality, and replicative senescence, cause low persistence of infused T cells, leading to the reduction of overall therapeutic efficacy in patients. New methods are needed to optimize the therapeutic efficacy of CAR T cells.

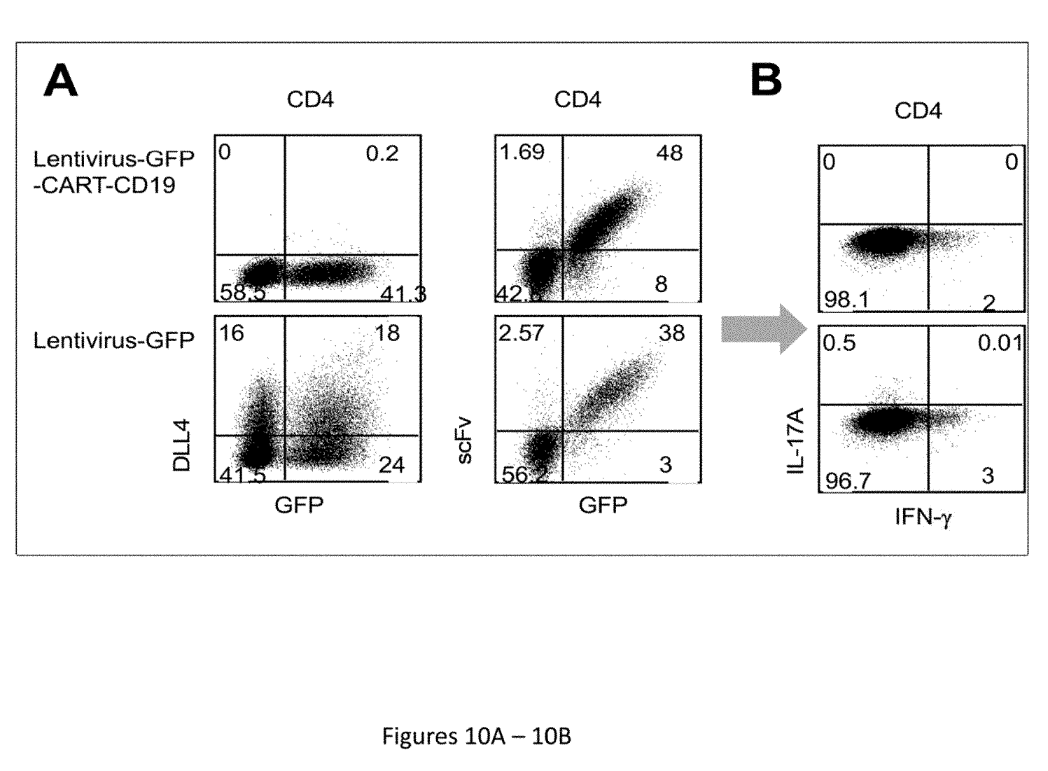

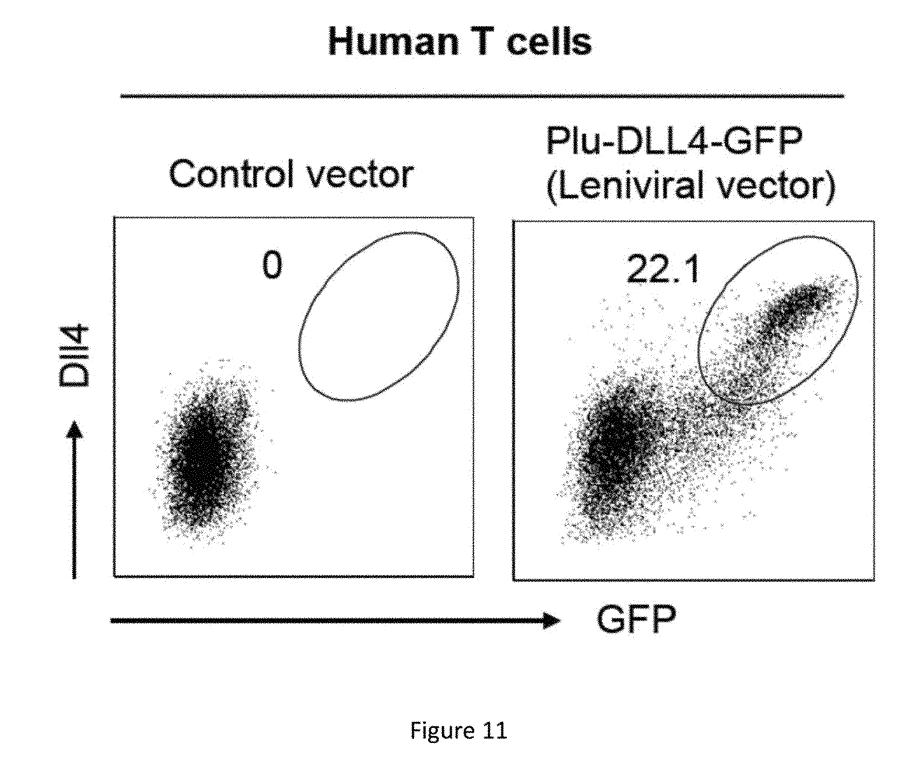

Given the important role of DC-derived DLL4 in regulating effector T cell responses, but without wishing to be bound by a particular theory, it was believed that DLL4hi DCs could be used for augmenting adoptive cellular therapy using naturally occurring and engineered T cells. In addition, engineering CART cells with DLL4 could potentially augment the persistence and function of T cells used for adoptive T cell therapy. CAR T cells were engineered with lentivirus encoding human DLL4. Seven days after viral introduction of DLL4, flow cytometry analysis showed a substantial proportion of CART-CD19 cells expressed high levels of DLL4 (FIG. 10A and FIG. 11). Further, T cells expressing DLL4 were able to regulate T cell immune response (FIG. 10A) and induced TH17 cells (FIG. 10B).

### Example 3

### DLL4 DC-Based Vaccination Results in the Induction and Expansion of Antigen-Specific CD4 T Cells and CD4 T Cells Producing High Levels of IFN-γ

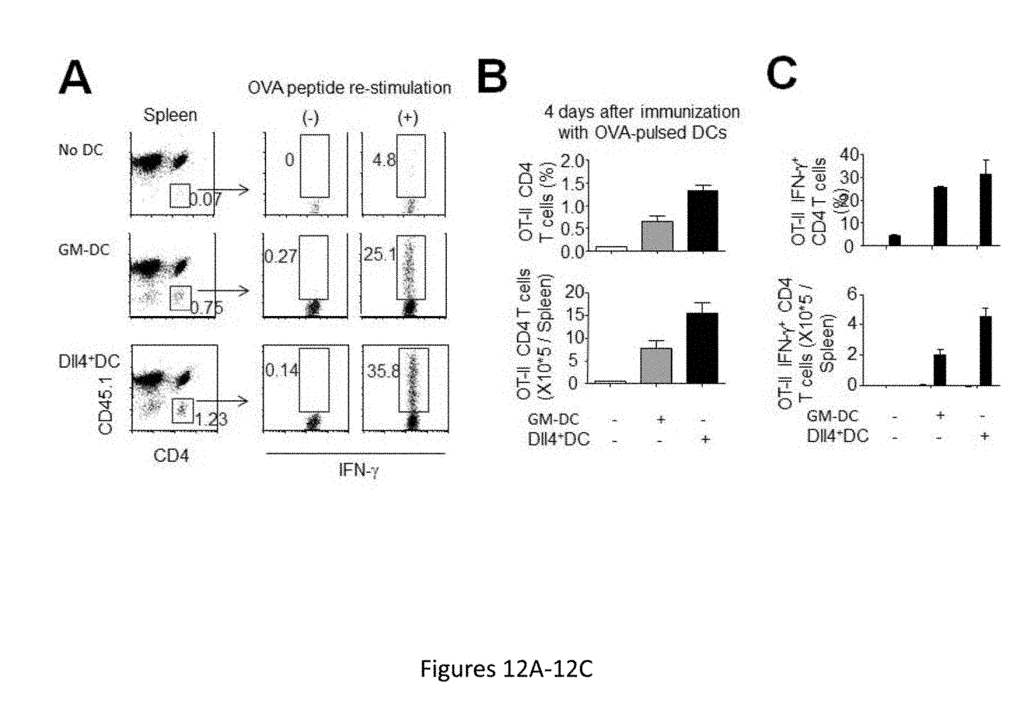

FIG. 12 shows the effect of DLL4+ DC vaccination on the induction and expansion of antigen-specific CD4 T cells compared to that of GM-DC vaccination. In this experiment, murine DLL4+ DCs and GM-DCs were generated from bone marrow, pulsed with OVA for 3 hours, and adoptively transferred into sub-lethally irradiated B6/SJL mice (CD45.1+) on day 0. OT-II specific CD4 T cells (CD45.2+) were transferred into these B6/SJL mice after radiation but before DC vaccination. DC vaccination was repeated on day+1 and day+2. Donor T cells were recovered at day 4 after vaccination to measure their expansion and cytokine production. DLL4^{hi} DC-based vaccination resulted in the induction and expansion of antigen-specific CD4 T cells and CD4 T cells producing high levels of IFN-γ as compared to GM-DCs.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A composition comprising a population of DLL4-expressing immune cells.

2. The composition of claim 1, wherein the immune cells comprise a nucleic acid molecule encoding DLL4.

3. (canceled)

4. The composition of claim 1, wherein the immune cells are selected from the group consisting of immature dendritic cells, mature dendritic cells, activated dendritic cells, T cells, natural killer T (NKT) cells and chimeric antigen receptor (CAR) T cells.

5. The composition of claim 4, wherein the immune cells are CAR T cells comprising a nucleic acid molecule encoding DLL4 and a nucleic acid molecule encoding a CAR.

6. The composition of claim 1, wherein the immune cells are antigen loaded, activated, DLL4-expressing dendritic cells (DCs).

7. A composition comprising a population of activated T cells for use in immunotherapy, wherein the T cells have been activated by antigen loaded, activated, DLL4-expressing dendritic cells (DCs) of claim 6.

8. A method of generating a DLL4-expressing, antigen loaded, activated dendritic cell (DC) of claim 6, comprising:
loading at least one antigen into a DC, wherein the DC is a bone marrow derived DC; and
activating the DC with Flt3L and at least one TLR, wherein the at least one TLR comprises at least one selected from the group consisting of LPS, R848, and a combination thereof.

9.-10. (canceled)

11. A method of generating a DLL4-expressing, antigen loaded, activated DC of claim 6, comprising:
loading at least one antigen into a DC;
genetically modifying the DC to express DLL4; and
activating the DC.

12. The method of claim 11, wherein the method of genetically modifying comprises providing a DNA-plasmid-based system or an mRNA-based system encoding DLL4 to the DC.

13.-15. (canceled)

16. A method of eliciting an immune response in a subject, the method comprising administering to the subject an effective amount of a population of DLL4-expressing immune cells of claim 1.

17. The method of claim 15, wherein the immune response is selected from the group consisting of a Th17 immune response and a Th1 immune response.

18. A method of providing anti-tumor immunity in a subject, the method comprising: administering to the subject an effective amount of antigen loaded, activated, DLL4-expressing DCs of claim 6.

19. The method of claim 18, wherein the DC is a bone marrow derived DC and further wherein the DC is activated with Flt3L and at least one TLR agonist, wherein the at least one TLR comprises at least one selected from the group consisting of LPS, R848, and a combination thereof.

20. (canceled)

21. The method of claim 18, wherein the DC is genetically modified to express DLL4.

22. A method of generating an activated T cell for use in immunotherapy, comprising contacting a naïve T cell with an antigen loaded, activated, DLL4-expressing DC of claim 6.

23. The method of claim 22, wherein the antigen loaded, activated, DLL4-expressing DC is a bone marrow derived DC and further wherein the DC is activated with Flt3L and at least one TLR agonist, wherein the at least one TLR comprises at least one selected from the group consisting of LPS, R848, and a combination thereof.

24. (canceled)

25. The method of claim 22, wherein the DC is genetically modified to express DLL4.

26. A method of stimulating a T cell-mediated immune response to a cell population or tissue in a subject, the method comprising: administering to the subject an effective amount of a T cell selected from the group consisting of a genetically modified T cell comprising a nucleic acid sequence encoding DLL4, a DLL4-expressing CAR T cell and an activated T cell wherein the T cell is activated according to the method of claim 22.

27. A method of treating cancer in a subject, the method comprising:
administering to the subject an effective amount of a genetically modified T cell comprising a nucleic acid sequence encoding DLL4, and a nucleic acid sequence encoding a CAR, wherein the CAR nucleic acid sequence comprises an antigen binding domain nucleic acid sequence, thereby treating cancer in the subject.

28. The method of claim 27, wherein the antigen binding domain nucleic acid targets CD19.


SEQUENCE LISTING (2 sequences)

SEQ ID NO: 1 - DNA, 2058 bp, Homo sapiens
atggcggcagcgtcccggagcgcctctggctgggcgctactgctgctggtggcactttggcagcagcgcgcggccggctccggcgtcttccagctgcagctgcaggagttcatcaacgagcgcggcgtactggccagtgggcggccttgcgagcccggctgccggactttcttccgcgtctgccttaagcacttccaggcggtcgtctcgcccggaccctgcaccttcgggaccgtctccacgccggtattgggcaccaactccttcgctgtccgggacgacagtagcggcggggggcgcaaccctctccaactgcccttcaatttcacctggccgggtaccttctcgctcatcatcgaagcttggcacgcgccaggagacgacctgcggccagaggccttgccaccagatgcactcatcagcaagatcgccatccagggctccctagctgtgggtcagaactggttattggatgagcaaaccagcaccctcacaaggctgcgctactcttaccgggtcatctgcagtgacaactactatggagacaactgctcccgcctgtgcaagaagcgcaatgaccacttcggccactatgtgtgccagccagatggcaacttgtcctgcctgcccggttggactggggaatattgccaacagcctatctgtctttcgggctgtcatgaacagaatggctactgcagcaagccagcagagtgcctctgccgcccaggctggcagggccggctgtgtaacgaatgcatcccccacaatggctgtcgccacggcacctgcagcactccctggcaatgtacttgtgatgagggctggggaggcctgttttgtgaccaagatctcaactactgcacccaccactccccatgcaagaatggggcaacgtgctccaacagtgggcagcgaagctacacctgcacctgtcgcccaggctacactggtgtggactgtgagctggagctcagcgagtgtgacagcaacccctgtcgcaatggaggcagctgtaaggaccaggaggatggctaccactgcctgtgtcctccgggctactatggcctgcattgtgaacacagcaccttgagctgcgccgactccccctgcttcaatgggggctcctgccgggagcgcaaccagggggccaactatgcttgtgaatgtccccccaacttcaccggctccaactgcgagaagaaagtggacaggtgcaccagcaacccctgtgccaacgggggacagtgcctgaaccgaggtccaagccgcatgtgccgctgccgtcctggattcacgggcacctactgtgaactccacgtcagcgactgtgcccgtaacccttgcgcccacggtggcacttgccatgacctggagaatgggctcatgtgcacctgccctgccggcttctctggccgacgctgtgaggtgcggacatccatcgatgcctgtgcctcgagtccctgcttcaacagggccacctgctacaccgacctctccacagacacctttgtgtgcaactgcccttatggctttgtgggcagccgctgcgagttccccgtgggcttgccgcccagcttcccctgggtggccgtctcgctgggtgtggggctggcagtgctgctggtactgctgggcatggtggcagtggctgtgcggcagctgcggcttcgacggccggacgacggcagcagggaagccatgaacaacttgtcggacttccagaaggacaacctgattcctgccgcccagcttaaaaacacaaaccagaagaaggagctggaagtggactgtggcctggacaagtccaactgtggcaaacagcaaaaccacacattggactataatctggccccagggcccctggggcgggggaccatgccaggaaagtttccccacagtgacaagagcttaggagagaaggcgccactgcggttacacagtgaaaagccagagtgtcggatatcagcgatatgctcccccagggactccatgtaccagtctgtgtgtttgatatcagaggagaggaatgaatgtgtcattgccacggaggtataa

SEQ ID NO: 2 - protein, 688 aa, Homo sapiens
MRNAMAAASRSASGWALLLLVALWQQRAAGSGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEKKVDRCTSNPCANGGQCLNRGPSRMCRCRPGFTGTYCELHVSDCARNPCAHGGTCHDLENGLMCTCPAGFSGRRCEVRTSIDACASSPCFNRATCYTDLSTDTFVCNCPYGFVGSRCEFPVGLPPSFPWVAVSLGVGLAVLLVLLGMVAVAVRQLRLRRPDDGSREAMNNLSDFQKDNLIPAAQLKNTNQKKELEVDCGLDKSNCGKQQNHTLDYNLAPGPLGRGTMPGKFPHSDKSLGEKAPLRLHSEKPECRISAICSPRDSMYQSVCLISEERNECVIATEV